(19) United States
(12) Patent Application Publication
(10) Pub. No.: US 2024/0375098 A1
(43) Pub. Date: Nov. 14, 2024

(54) MICROFLUIDIC CHIP AND DETECTION SYSTEM, DETECTION METHOD AND MANUFACTURING METHOD THEREFOR

(71) Applicants: Beijing BOE Sensor Technology Co., Ltd., Beijing (CN); BOE TECHNOLOGY GROUP CO., LTD., Beijing (CN)

(72) Inventors: Bolin FAN, Beijing (CN); Le GU, Beijing (CN); Yingying ZHAO, Beijing (CN); Yongjia GAO, Beijing (CN); Li YANG, Beijing (CN)

(21) Appl. No.: 18/577,846

(22) PCT Filed: Jun. 30, 2022
(86) PCT No.: PCT/CN2022/102853

§ 371 Date: Jan. 9, 2024

(51) Int. Cl. B01L 3/00; G01R 31/28

(52) U.S. Cl. CPC B01L 3/502707; G01R 31/28

(57) ABSTRACT

A microfluidic chip and a detection system, a detection method and a manufacturing method therefor are provided. A crimping device for the microfluidic chip includes a cover plate, a bottom plate and at least one probe assembly. The bottom plate is assembled with the cover plate. The bottom plate is provided with a carrying recess. A mouth of the carrying recess faces the cover plate, and a bottom of the carrying recess is provided with an opening. The probe assembly includes a plurality of probes. The probe assembly is fixedly connected with the bottom of the carrying recess. Ends, proximate to the cover plate, of the plurality of probes are configured to be in contact with the microfluidic chip; and ends, away from the cover plate, of the plurality of probes pass through the opening.

### CROSS-REFERENCE TO RELATED APPLICATION

This application is a national phase entry under 35 USC 371 of International Patent Application No. PCT/CN2022/102853 filed on Jun. 30, 2022, which is incorporated herein by reference in its entirety.

### TECHNICAL FIELD

The disclosure relates to the technical field of display technologies, in particular to a microfluidic chip and a detection system, a detection method and a manufacturing method thereof.

### BACKGROUND

Microfluidic technologies (microfluidics) refer to technologies for processing or controlling tiny (nanoliters (nL) to microliters (μL) in volume) fluids using microchannels (tens to hundreds of micrometers in size). Microfluidic chips are main platforms for realizing microfluidic technologies. The microfluidic chips have characteristics such as parallel sample collection and treatment, high integration, high flux, quick analysis, low power consumption, low material consumption and low contamination. The microfluidic technologies may be applied to the fields such as biological gene engineering, disease diagnosis and drug research, cell analysis, environmental monitoring and protection, health quarantine and judicial identification, and mainly relates to uniform mixing and transportation of trace reagent samples. The transportation of the sample is one of basic steps of biochemical detection.

In recent years, the microfluidic technologies have been rapidly developed, and have higher and higher requirements for various performances of the microfluidic chips. Reliability and stability of sample transportation are one of key performances for the microfluidic chips to realize a target biochemical process, and an optimization of this performance has an important significance for the development of the fields such as biomedicine, drug diagnosis, food hygiene, environmental monitoring and molecular biology.

### SUMMARY

In an aspect, a crimping device for a microfluidic chip is provided. The crimping device includes a cover plate, a bottom plate and at least one probe assembly.

The bottom plate is assembled with the cover plate. The bottom plate is provided with a carrying recess. A mouth of the carrying recess faces the cover plate, and a bottom of the carrying recess is provided with an opening. The probe assembly includes a plurality of probes. The probe assembly is fixedly connected with the bottom of the carrying recess. Ends, proximate to the cover plate, of the plurality of probes are configured to be in contact with the microfluidic chip; and ends, away from the cover plate, of the plurality of probes pass through the opening.

In some embodiments, the probe assembly further includes a needle module. The needle module is fixedly connected with the bottom of the carrying recess. The needle module is provided therein with a plurality of mounting holes. A probe of the plurality of mounting holes passes through a respective mounting hole, and is fixed in the mounting hole.

In some embodiments, the bottom of the carrying recess includes a plurality of carrying portions. The plurality of carrying portions are arranged at intervals around a wall of the carrying recess. At least one of the plurality of carrying portions is raised with respect to the wall. The probe assembly is disposed between two adjacent carrying portions. Two ends of the probe assembly are fixedly connected with the two adjacent carrying portions, respectively.

In some embodiments, the at least one probe assembly includes a plurality of probe assemblies. At least one of the plurality of probe assemblies is a bonding probe assembly. The bonding probe assembly is disposed proximate to a wall of the carrying recess. Ends, proximate to the cover plate, of a plurality of probes of the bonding probe assembly are configured to be in contact with bonding electrodes of the microfluidic chip.

In some embodiments, at least one of the plurality of probe assemblies is a driving probe assembly. With respect to the bonding probe assembly, the driving probe assembly is disposed away from the wall of the carrying recess. Ends, proximate to the cover plate, of a plurality of probes of the driving probe assembly are configured to be in contact with driving electrodes of the microfluidic chip.

In some embodiments, the bottom of the carrying recess is further provided therein with a first through hole. The crimping device further includes a grounding probe. The grounding probe is fixedly connected with the bottom of the carrying recess. An end, proximate to the cover plate, of the grounding probe is configured to be in contact with the microfluidic chip; and an end, away from the cover plate, of the grounding probe passes through the first through hole.

In some embodiments, in a case where the crimping device further comprises the grounding probe, the plurality of probes and the grounding probe have elasticity in respective length extension directions thereof. An elastic deformation range of the grounding probe is greater than an elastic deformation range of at least one of the plurality of probes.

In some embodiments, in a case where the crimping device further comprises the press-fit structure, the press-fit structure includes a press-fit plate and an elastic member. The elastic member is located between the press-fit plate and the cover plate. An end of the elastic member is connected with the press-fit plate, and another end of the elastic member is connected with the cover plate. In the case where the cover plate is buckled with the bottom plate, an orthographic projection of the press-fit plate on the reference surface at least partially overlaps with the orthographic projection of the probe assembly on the reference surface.

In some embodiments, the crimping device further includes hinge structures. Each hinge structure includes a first hinge and a second hinge which are movably connected with each other. The first hinge is fixedly connected with the cover plate. The second hinge is fixedly connected with the bottom plate.

In some embodiments, the crimping device further includes hinge structures. The hinge structures include first hinges and second hinges movably connected with each other. The first hinges are fixedly connected with the cover plate, and the second hinges are fixedly connected with the bottom plate.

In some embodiments, the crimping device further includes a snap-fit structure. The snap-fit structure includes a buckle and a latching slot. The buckle is fixedly connected with one of the cover plate and the bottom plate. The latching slot is provided in another one of the cover plate and the bottom plate. The buckle is capable of being snap-fitted to the latching slot. The hinge structures and the snap-fit structure are respectively arranged on two opposite sides of the cover plate and the bottom plate.

In another aspect, a detection apparatus for a microfluidic chip is provided. The detection apparatus includes a circuit board, a processor and the crimping device as described in any one of the above embodiments.

The circuit board is disposed on a side, away from the cover plate, of the bottom plate of the crimping device. The circuit board includes a substrate and a plurality of pads disposed on the substrate. A pad of the plurality of pads is in contact with a respective probe of the crimping device. The processor is disposed on the circuit board, and electrically connected with the plurality of pads. The processor is configured to transmit detection signals to probes in contact with the pads through the pads, receive feedback signals from the probes, and process the feedback signals.

In some embodiments, the plurality of pads are disposed on a side of the substrate proximate to the bottom plate, and the processor is disposed on a side of the substrate away from the bottom plate.

In some embodiments, the crimping device includes a grounding probe. The circuit board further includes a grounding pad. The grounding pad is in contact with the grounding probe.

In some embodiments, the circuit board further includes a resistor. The resistor is connected in parallel with the processor.

In some embodiments, a resistance of the resistor is less than or equal to a resistance of the processor.

In yet another aspect, a detection system for a microfluidic chip is provided. The detection system includes an industrial personal computer and the detection apparatus as described in any one of the above embodiments. The industrial personal computer is electrically connected with the circuit board of the detection apparatus.

In yet another aspect, a method for detecting a microfluidic chip is provided. In the method, a detection is performed by using the detection apparatus as described in any one of the above embodiments.

The method includes: placing the microfluidic chip in the carrying recess of the bottom plate of the detection apparatus, the microfluidic chip including a plurality of electrodes, and an electrode of the plurality of electrodes being in contact with a respective probe of the detection apparatus; detecting whether two electrodes, insulated from each other, of the plurality of electrodes are shorted to each other; and/or the microfluidic chip including a plurality of circuits each having at least two of the plurality of electrodes connected in series, detecting whether a circuit of the plurality of circuits is broken.

In some embodiments, detecting whether the two electrodes, insulated from each other, of the plurality of electrodes are shorted to each other, includes: combining the plurality of electrodes pairwise to obtain a plurality of electrode pairs, two electrodes of each electrode pair being insulated from each other, for any two electrode pairs, two electrodes of one electrode pair being not completely same as two electrodes of another electrode pair; detecting whether the two electrodes of each electrode pair are shorted to each other; recording positions of two electrodes of an electrode pair according to that the two electrodes of the electrode pair are shorted to each other; and determining that the microfluidic chip has no short-circuit fault according to that the two electrodes of each of the plurality of electrode pairs are not shorted to each other.

In some embodiments, detecting whether the two electrodes of each electrode pair are shorted to each other, includes: detecting a voltage between the two electrodes of the electrode pair; determining that two electrodes of an electrode pair are shorted to each other according to that a voltage is less than or equal to a threshold voltage; and determining that two electrodes of an electrode pair are not shorted to each other according to a voltage is greater than the threshold voltage.

In some embodiments, the detection apparatus includes a processor and a resistor connected in parallel with the processor. The threshold value is:

$${V}_{1} = \frac{{R}_{1} V}{{R}_{1} + \frac{{R}_{2} {R}_{3}}{{R}_{2} + {R}_{3}}} ,$$

where V₁ is the threshold voltage, V is a power supply voltage of the detection apparatus, R₁ is a resistance between two electrodes of an electrode pair in a case where the two electrodes are shorted to each other, R₂ is a resistance of the processor, and R₃ is a resistance of the resistor.

In some embodiments, detecting whether the circuit of plurality of circuits is broken, includes: detecting a voltage between two electrodes, at two ends, of at least two electrodes connected in series with each other of the circuit; determining that the circuit is broken, and recording positions of the at least two electrodes connected in series with each other of the circuit, according to that the voltage is greater than a threshold voltage; and determining that the circuit is non-broken according to the voltage is less than or equal to the threshold voltage.

In some embodiments, the plurality of electrodes includes two fool-proof electrodes, the two fool-proof electrodes are electrically connected with each other, and the two fool-proof electrodes are arranged asymmetrically with respect to a setting center line of the microfluidic chip. The microfluidic chip includes a first side edge and a second side edge that are opposite to each other. The setting center line is a center line parallel to the first side edge and the second side edge of the microfluidic chip.

The method further includes: detecting whether two electrodes at target positions are shorted to each other, the target positions being positions where the two fool-proof electrodes are located in a case where the microfluidic chip is placed in the detection apparatus in a correct position; determining that the two electrodes at the target positions are not the two fool-proof electrodes according to that the two electrodes at the target positions are not shorted to each other, and reversing positions of the first side edge and the second side edge of the microfluidic chip; and determining that the two electrodes at the target positions are the two foolproof electrodes and the microfluidic chip is places in the correct position, according to that the two electrodes at the target positions are shorted to each other.

In yet another aspect, a method for manufacturing a microfluidic chip is provided. The method includes: forming a first substrate and a second substrate, the first substrate including a plurality of electrodes; placing the first substrate in a detection apparatus, and detecting the first substrate by the method as described in any one of the above embodiments; forming a dielectric layer on the plurality of electrodes of the first substrate according to a detection result that no fault exists; detecting the first substrate on which the dielectric layer has been formed; forming a first hydrophobic layer on the dielectric layer according to a detection result that no fault exists; detecting the first substrate on which the first hydrophobic layer has been formed; and assembling the first substrate on which the first hydrophobic layer has been formed with the second substrate, according to a detection result that no fault exists.

In yet another aspect, a microfluidic chip is provided. The microfluidic chip includes a first substrate and a second substrate.

The first substrate includes two fool-proof electrodes. The two fool-proof electrodes are electrically connected with each other, and arranged asymmetrically with respect to a setting center line of the microfluidic chip. The microfluidic chip includes a first side edge and a second side edge that are opposite to each other. The setting center line passes through midpoints of lines connecting the first side edge with the second side edge at arbitrary positions, and is parallel to the first side edge and the second side edge. The second substrate is assembled with the array substrate.

In some embodiments, the first substrate includes a plurality of driving electrodes and a plurality of bonding electrodes. The bonding electrodes are electrically connected with at least one of the plurality of driving electrodes. Two bonding electrodes of the plurality of bonding electrodes serve as the two fool-proof electrodes. The two fool-proof electrodes are both disposed on a side of the setting center line proximate to the first side edge.

In some embodiments, the first substrate includes a plurality of driving electrodes and a plurality of bonding electrodes. The bonding electrodes are electrically connected with at least one of the driving electrodes. The first substrate further includes a temperature sensor. The temperature sensor is disposed adjacent to and insulated from at least one driving electrode. The temperature sensor is electrically connected with the two fool-proof electrodes.

In some embodiments, the temperature sensor is a wire wound resistor. The wound wire resistor is a wire extending in a broken line pattern, and two ends of the wire are electrically connected with the two fool-proof electrodes, respectively.

In some embodiments, a fool-proof electrode of the two fool-proof electrodes is grounded.

In some embodiments, the second substrate is provided with a second through hole. An orthogonal projection of the second through hole on the first substrate at least partially overlaps with the grounded fool-proof electrode.

In some embodiments, the first substrate further includes a plurality of driving electrodes, a plurality of bonding electrodes and a plurality of detection electrodes. At least one driving electrode is electrically connected with both a bonding electrode and a detection electrode. The bonding electrode and the detection electrode electrically connected with the at least one driving electrode are configured to be electrically connected with a detection apparatus to realize an open-circuit detection.

In some embodiments, the second substrate is provided with at least one second window. The second window is configured to expose the plurality of bonding electrodes and/or the plurality of detection electrodes of the first substrate.

### BRIEF DESCRIPTION OF THE DRAWINGS

In order to describe technical solutions in the present disclosure more clearly, accompanying drawings to be used in some embodiments of the present disclosure will be introduced briefly below. However, the accompanying drawings to be described below are merely accompanying drawings of some embodiments of the present disclosure, and a person having ordinary skill in the art can obtain other drawings according to these accompanying drawings. In addition, the accompanying drawings in the following description may be regarded as schematic diagrams, but are not limitations on an actual size of a product, an actual process of a method and an actual timing of a signal involved in the embodiments of the present disclosure.

FIG. 1 is a section of a microfluidic chip, in accordance with some embodiments;

FIG. 2 is a structural diagram of a detection system for a microfluidic chip, in accordance with some embodiments;

FIG. 3 is a structural diagram of a detection apparatus for a microfluidic chip, in accordance with some embodiments;

FIG. 4 is an exploded view of a detection apparatus for the microfluidic chip, in accordance with some embodiments;

FIG. 5 is a structural diagram of a bottom plate of the microfluidic chip, in accordance with some embodiments;

FIG. 6 is a structural diagram of a cover plate of the microfluidic chip, in accordance with some embodiments;

FIG. 7 is a structural diagram of a probe assembly of the microfluidic chip, in accordance with some embodiments;

FIG. 8 is a structural diagram of a probe, in accordance with some embodiments;

FIG. 9 is a structural diagram of a structure obtained by assembling probe assemblies to a bottom plate, in accordance with some embodiments;

FIG. 10 is a structural diagram of another structure obtained by assembling probe assemblies to a bottom plate, in accordance with some embodiments;

FIG. 11 is a structural diagram of a bottom plate of another microfluidic chip, in accordance with some embodiments;

FIG. 12 is an exploded view of a press-fit device of a microfluidic chip, in accordance with some embodiments;

FIG. 13 is a structural diagram of a structure obtained by assembling probe assemblies and a press-fit structure to a bottom plate, in accordance with some embodiments;

FIG. 14 is a structural diagram of hinge structures, in accordance with some embodiments;

FIG. 15 is a front view of a circuit board, in accordance with some embodiments;

FIG. 16 is a rear view of the circuit board, in accordance with some embodiments;

FIG. 17 is a flowchart of a method for detecting a microfluidic chip, in accordance with some embodiments;

FIG. 18 is a flowchart of another method for detecting a microfluidic chip, in accordance with some embodiments;

FIG. 19 is a flowchart of yet another method for detecting a microfluidic chip, in accordance with some embodiments;

FIG. 20 is a flowchart of yet another method for detecting a microfluidic chip, in accordance with some embodiments;

FIG. 21 is a flowchart of yet another method for detecting a microfluidic chip, in accordance with some embodiments;

FIG. 22 is a flowchart of a method for manufacturing a microfluidic chip, in accordance with some embodiments;

FIG. 23 is a diagram showing a manufacturing process for a method for manufacturing a microfluidic chip, in accordance with some embodiments;

FIG. 24 is a structural diagram of a first substrate of a microfluidic chip, in accordance with some embodiments;

FIG. 25 is a structural diagram of a first substrate of another microfluidic chip, in accordance with some embodiments;

FIG. 26 is an enlarged view of a structure at a position where a region B is located in FIG. 25;

FIG. 27 is a structural diagram of a first substrate of yet another microfluidic chip, in accordance with some embodiments; and

FIG. 28 is a structural diagram of a second substrate of a microfluidic chip, in accordance with some embodiments.

### DETAILED DESCRIPTION

Technical solutions in some embodiments of the present disclosure will be described clearly and completely below with reference to the accompanying drawings. However, the described embodiments are merely some but not all embodiments of the present disclosure. All other embodiments obtained by a person of ordinary skill in the art based on the embodiments of the present disclosure shall be included in the protection scope of the present disclosure.

Unless the context requires otherwise, throughout the description and the claims, the term “comprise” and other forms thereof such as the third-person singular form “comprises” and the present participle form “comprising” are construed as an open and inclusive meaning, i.e., “including, but not limited to”. In the description of the specification, the terms such as “one embodiment”, “some embodiments”, “exemplary embodiments”, “example”, “specific example” or “some examples” are intended to indicate that specific features, structures, materials or characteristics related to the embodiment(s) or example(s) are included in at least one embodiment or example of the present disclosure. Schematic representation of the above terms does not necessarily refer to the same embodiment(s) or example(s). In addition, the specific features, structures, materials or characteristics may be included in any one or more embodiments or examples in any suitable manner.

Hereinafter, the terms such as “first” and “second” are used for descriptive purposes only, but are not to be construed as indicating or implying the relative importance or implicitly indicating the number of indicated technical features. Thus, the features defined with “first” and “second” may explicitly or implicitly include one or more of the features. In the description of the embodiments of the present disclosure, the term “a plurality of/the plurality of” means two or more unless otherwise specified.

Some embodiments may be described using the terms “coupled” and “connected with” and their derivatives. For example, the term “connected with” may be used in the description of some embodiments to indicate that two or more components are in direct physical or electrical contact with each other. The embodiments disclosed herein are not necessarily limited to the content herein.

The phrase “at least one of A, B and C” has a same meaning as the phrase “at least one of A, B or C”, and they both include the following combinations of A, B and C: only A, only B, only C, a combination of A and B, a combination of A and C, a combination of B and C, and a combination of A, B and C.

The phrase “A and/or B” includes the following three combinations: only A, only B, and a combination of A and B.

The phrase “applicable to” or “configured to” as used herein indicates an open and inclusive expression, which does not exclude devices that are applicable to or configured to perform additional tasks or steps.

In addition, the use of the phrase “based on” is meant to be open and inclusive, since a process, step, calculation or other action that is “based on” one or more of the stated conditions or values may, in practice, be based on additional conditions or values exceeding those stated.

Terms such as “about”, “substantially” or “approximately” as used herein include a stated value and an average value within an acceptable range of deviation of a particular value. The acceptable range of deviation is determined by a person of ordinary skill in the art in view of the measurement in question and the error associated with the measurement of a particular quantity (i.e., the limitations of the measurement system).

In the description of the present disclosure, it will be understood that orientations or positional relationships indicated by terms “center”, “longitudinal”, “transverse”, “length”, “width”, “vertical”, “horizontal”, “inner”, “outer”, etc. are based on orientations or positional relationships shown in the accompanying drawings, which are merely to facilitate and simplify the description of the present disclosure, but not to indicate or imply that the referred devices or elements must have a particular orientation, or must be constructed and operated in a particular orientation. Therefore, they should not be construed as limitations to the present disclosure.

It will be understood that, in a case where a layer or a component is described as being on another layer or a substrate, it may be that the layer or the component is directly on the another layer or the substrate, or that intermediate layer(s) exist between the layer the component and the another layer or the substrate.

Exemplary embodiments are described herein with reference to sectional views and/or plan views as idealized exemplary drawings. In the accompanying drawings, thicknesses of layers and areas of regions are enlarged for clarity. Thus, variations in shape with respect to the accompanying drawings due to, for example, manufacturing technologies and/or tolerances may be envisaged. Therefore, the exemplary embodiments should not be construed as being limited to the shapes of the regions shown herein, but including shape deviations due to, for example, manufacturing. For example, an etched region shown in a rectangular shape generally has a feature of being curved. Therefore, the regions shown in the accompanying drawings are schematic in nature, and their shapes are not intended to show actual shapes of the region in a device, and are not intended to limit the scope of the exemplary embodiments.

A microfluidic system integrates complex laboratory functions into a single analytical apparatus or a single chip by constructing microdevices, and thus realizes miniaturization and integration of an analytical system.

In some embodiments, basic operation units such as preparation, reaction, separation and detection of a sample to be detected are integrated into a centimeter-level chip to form a microfluidic chip. The microfluidic chip is provided therein with microchannels. The microfluidic system realizes accurate control and operation of the sample to be detected in a microchannel by applying driving forces to the microfluidic chip.

It will be noted that, the sample to be detected may be a liquid substance. For example, the sample to be detected is a blood sample. Molecules to be detected in the blood sample are, for example, hemoglobin, platelets or pathogenic cells. In a microfluidic process, the liquid substance as the sample to be detected is placed in a microfluidic chip in a form of a droplet. The following embodiments are all described by taking an example where the sample to be detected is a droplet.

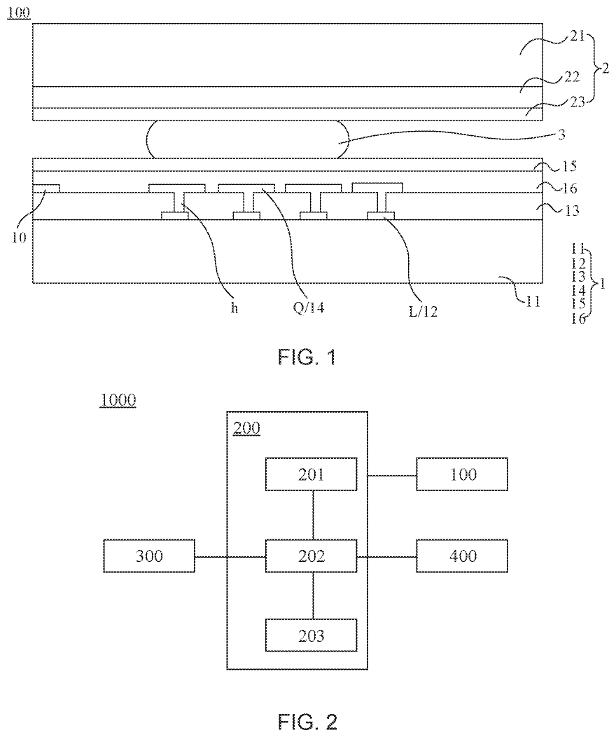

FIG. 1 shows a sectional structure of a microfluidic chip 100. As shown in FIG. 1, in some embodiments, the microfluidic chip 100 includes a first substrate 1 and a second substrate 2 that are assembled with each other.

The first substrate 1 and the second substrate 2 are arranged opposite to and spaced from each other. A droplet 3 is placed in a gap between the first substrate 1 and the second substrate 2. By changing a voltage between the first substrate 1 and the second substrate 2, a contact angle of the droplet 3 placed between the first substrate 1 and the second substrate 2 may be changed, so that phenomena such as deformation and displacement of the droplet 3 occur, and control of the droplet 3 is realized.

In some embodiments, with reference to FIG. 1, the second substrate 2 includes a second base 21 and a common electrode layer 22 and a second hydrophobic layer 23 that are sequentially disposed on the second base 21. The second base 21 is away from the first substrate 1 with respect to the second hydrophobic layer 23.

In some embodiments, the common electrode layer 22 is formed by a continuous transparent conductive indium tin oxide (ITO) layer.

For example, as a common electrode of the microfluidic chip 100, the common electrode layer 22 is grounded so as to provide a stable low-level voltage to the microfluidic chip 100.

It will be noted that, the common electrode layer 22 may also be disposed in the first substrate 1. Embodiments of the present disclosure only illustrate an example where the common electrode layer 22 is disposed in the second substrate 2, which does not limit a specific structure of the microfluidic chip 100.

In some embodiments, with reference to FIG. 1, the first substrate 1 includes a first base 11 and a first conductive layer 12, an insulating layer 13, a second conductive layer 14 and a first hydrophobic layer 15 that are sequentially disposed on the first base 11. The first base 11 is away from the second substrate 2 with respect to the first hydrophobic layer 15. A channel of the droplet 3 is formed between the first hydrophobic layer 15 and the second hydrophobic layer 23, which allows the droplet 3 to smoothly flow.

In some embodiments, the second conductive layer 14 is provided therein with a plurality of driving electrodes Q distributed in an array according to a preset electrode pattern (with reference to a rectangular channel LP shown in FIG. 24). The driving electrodes Q are electrically connected with a driving power supply to provide driving voltages for the microfluidic chip 100.

In some embodiments, with reference to FIG. 1, orthographic projections of the plurality of driving electrodes Q on the first base 11 are located within an orthographic projection of the common electrode layer 22 on the first base 11. That is, the plurality of driving electrodes Q have overlapping areas with respect to the common electrode layer 22.

In a case where a driving electrode Q is energized, wettability of the droplet 3 is changed due to an action of an electric field between the common electrode layer 22 and the energized driving electrode Q. This results in that, a contact angle of the droplet 3 on the driving electrode Q in a case where a voltage is applied to the driving electrode Q is different from a contact angle of the droplet 3 on the driving electrode Q in a case where no voltage is applied to the driving electrode Q, so that a pressure difference is generated inside the droplet 3, and the droplet 3 is driven to move in a direction of driving electrodes Q with applied voltages due to an action of pressure differences. Driving voltages are applied to different driving electrodes Q according to a time sequence, so that the droplet 3 can be driven to move along a preset path, and the control of the droplet 3 is realized.

For example, the driving electrodes Q are made of metal or other conductive materials. For example, the driving electrodes Q may be made of Mo, ITO or other materials.

In some embodiments, with reference to FIG. 1, the first conductive layer 12 includes a plurality of signal lines L made of metal.

Alternatively, the signal lines L are made of metal Mo. The signal lines L are configured to be electrically connected with the driving electrodes Q to transmit driving voltages to the driving electrodes Q.

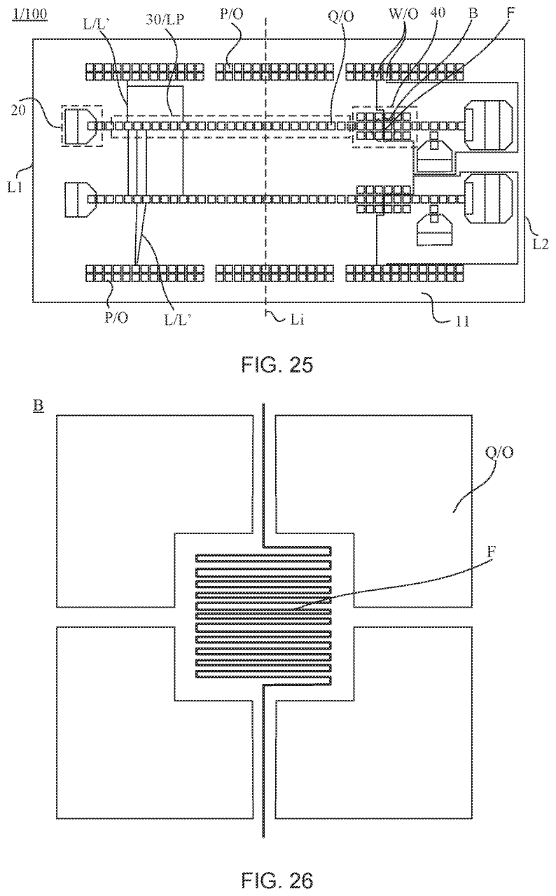

In some embodiments, with reference to FIG. 1, the plurality of signal lines L are electrically connected with the plurality of driving electrodes Q in a one-to-one correspondence, so that the plurality of driving electrodes Q are controlled independently of each other. Alternatively, in some embodiments, a signal line L may be electrically connected with at least two non-adjacent driving electrodes Q in a same flow channel (with reference to FIGS. 24 and 25). In this way, a number of the signal lines L is reduced, and a wiring space of the signal lines L is saved, while adjacent driving electrodes Q are controlled independently of each other.

In some embodiments, with reference to FIG. 1, the first conductive layer 12 and the second conductive layer 14 are provided therebetween with an insulating layer 13. The insulating layer 13 is provided with via holes h. The signal lines L in the first conductive layer 12 are electrically connected with the driving electrodes Q of the second conductive layer 14 through the via holes h. Alternatively, the first conductive layer 12 may be located between the second conductive layer 14 and the first base 11, or the second conductive layer 14 may be located between the first conductive layer 12 and the first base 11.

In some embodiments, with reference to FIG. 1, the first substrate 1 further includes a dielectric layer 16 disposed between the second conductive layer 14 and the first hydrophobic layer 15. The dielectric layer 16 is used for promoting charge accumulation and increasing electric field strength, which ensures that the microfluidic chip 100 may easily drive the droplet 3 without breakdown.

Depending on a material, different methods such as vapor deposition (of parylene, silicon nitride or amorphous fluoropolymer), thermal growth (of silicon dioxide) and spin coating (of polydimethylsiloxane or photoresist) may be selected to form the dielectric layer 16.

Alternatively, the dielectric layer 16 may adopt a polyimide (PI) film having a dielectric constant of 3.2.

In some embodiments, with reference to FIG. 1, the first hydrophobic layer 15 and the second hydrophobic layer 23 are in direct contact with the droplet 3. The first hydrophobic layer 15 and the second hydrophobic layer 23 are a fluoropolymer (polytetrafluoroethylene), and used for reducing a surface energy when during diving of the droplet 3.

In some embodiments, the first base 11 and the second base 21 may be made of glass with strong chemical inertness or a printed circuit board.

In some embodiments, with reference to FIG. 1, the first substrate 1 further includes a grounding electrode 10.

The grounding electrode 10 is grounded, so static electricity in the first substrate 1 are conducted out, which avoids an occurrence of electrostatic breakdown and then avoids an influence on a service life of the first substrate 1.

Alternatively, the grounding electrode 10 is composed of a 1 mm.times.1 mm (1 mm×1 mm) square electrode, and a size of the square electrode and a number of square electrodes may be adjusted depending on needs.

In some embodiments, the first substrate 1 further includes a plurality of bonding electrodes P (with reference to FIG. 24). At least one driving electrode Q may be electrically connected with a bonding electrode P through a signal line L.

The bonding electrode P is configured to be bonded to the driving power supply, so that a voltage of the driving power supply can be transmitted to the at least one driving electrode Q electrically connected with the bonding electrode P through the bonding electrode P, thereby realizing control of a droplet located between the driving electrode Q and the common electrode layer 22.

For example, the plurality of bonding electrodes P may be disposed in the second conductive layer 14, and the first hydrophobic layer 15 and the dielectric layer 16 are hollowed out at positions corresponding to the plurality of bonding electrodes P to expose the bonding electrodes P, so that the bonding electrodes P are connected with the driving power supply through bonding.

Due to a larger advantage in cost, passive digital microfluidic chips acts as a mainstream chip scheme in current commercial products of microfluidic chips 100. For a digital microfluidic chip 100 with high flux, reliability and stability of transportation of a sample to be detected are one of keys for the chip to realize a target biochemical process. Especially, in a biological or chemical micro-total analysis system with high integration, high performance and complex operation, the droplet 3 needs to be highly precisely controlled, and thus requirements for the reliability and stability of the transportation of the droplet 3 are higher.

However, as can be seen from the embodiments described above, the first substrate 1 of the microfluidic chip 100 is formed by stacking a plurality of layers of metal layers (such as the first conductive layer 12 and the second conductive layer 14) and the insulating layer 13, and such a structure is fine; and then an attachment of the dielectric layer 16 and a process of assembling the first substrate 1 with the second substrate 2 need to be completed at different working stations, and processes are numerous. In these processes, it is prone to a short-circuit failure or an open-circuit failure between the driving electrodes Q of the microfluidic chip 100 due to static electricity or pressure. Consequently, not only does a product yield of microfluidic chips 100 at a client side decrease, but also reliability of microfluidic chips 100 at an application side is significantly reduced, which limits a further development of microfluidic chips 100.

In order to solve the above technical problems, embodiments of the present disclosure provide a detection system 1000 for the microfluidic chip 100. The method is conductive to screening out microfluidic chip(s) 100 subject to a short-circuit failure or an open-circuit failure, and then improving the product yield.

As shown in FIG. 2, the detection system 1000 includes a detection apparatus 200 and an industrial personal computer 300.

The industrial personal computer 300 is electrically connected with a circuit board 202 of the detection apparatus 200.

For example, the circuit board 202 is disposed therein with an industrial personal computer interface. The industrial personal computer 300 is electrically connected with the circuit board 202 through the industrial personal computer socket.

For example, the industrial personal computer 300 is electrically connected with the circuit board 202 by a dupont line, so that power supply and data transmission of the industrial personal computer 300 are realized.

For example, the industrial personal computer 300 is configured to start or shut down an entire detection program. For example, after being powered on, the industrial personal computer 300 displays a detection button; and after the detection button is clicked, the detection apparatus 200 starts to detect the microfluidic chip 100.

For example, the industrial personal computer 300 is further configured to record a detection result (e.g., a short-circuit fault, an open-circuit fault or no fault) of the detection apparatus 200, and display positions of corresponding faulty electrodes of the microfluidic chip 100 according to the detection result.

For example, after the detection button is clicked, the detection apparatus 200 numbers electrodes to be detected of the microfluidic chip 100 in sequence. For example, in a case where there are n electrodes to be detected, numbers thereof are 1, 2, 3 . . . (n−1) and n in sequence.

After the detection apparatus 200 completes the detection, the industrial personal computer 300 displays detection results, and displays numbers corresponding to electrodes to be detected with a fault (a short-circuit fault or an open-circuit fault), so that positions of the electrodes to be detected with the fault are determined. For example, in a case where the industrial personal computer 300 displays that a detection result “short circuits exist”, the industrial personal computer 300 further displays numbers of corresponding short-circuited electrodes, such as “2 is shorted to 5, and 10 is shorted to 111”; and in a case where there is no short-circuit fault or no open-circuit fault, the industrial personal computer 300 displays “no fault exists” or “all channels are OK”.

It will be noted that, the “electrode to be detected” may include at least one of the driving electrodes Q, the bonding electrodes P or the grounding electrode 10. For example, when detecting whether a circuit in which a bonding electrode P is located is shorted to a circuit in which another bonding electrode P is located, the two bonding electrodes P may serve as electrodes to be detected to perform the short-circuit detection.

For example, the industrial personal computer 300 may be a box-type industrial personal computer, a rack-mount industrial personal computer, a panel-type industrial personal computer, etc.

For example, the industrial personal computer 300 has a display screen, and the display screen may display information such as detection results. For example, the display screen may be a touch display screen.

In some embodiments, as shown in FIG. 2, the detection system 1000 may further include a power supply 400. The power supply 400 is configured to provide power supply signals to the detection apparatus 200 and the industrial personal computer 300 so as to drive the detection system 1000 to run.

For example, the power supply 400 is electrically connected with the circuit board 202 of the detection apparatus 200.

For example, the circuit board 202 is provided therein with a power supply interface, and the power supply 400 is electrically connected with the circuit board 202 through the power supply socket.

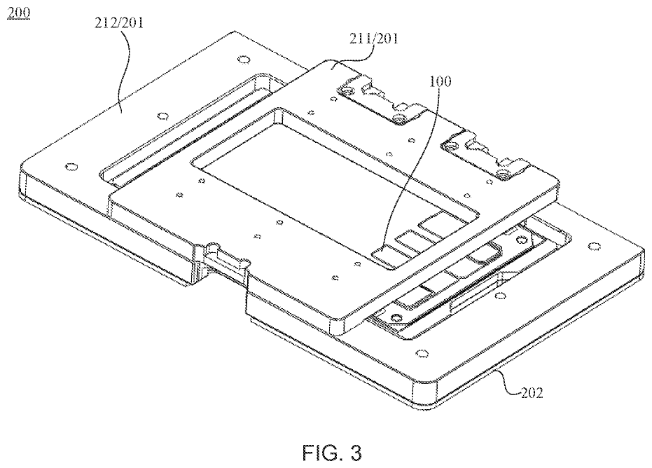

In some embodiments, as shown in FIGS. 2, 3 and 4, the detection apparatus 200 includes a crimping device 201, the circuit board 202 and a processor 203.

The crimping device 201 is configured to fix the microfluidic chip 100 during detection, which avoids a large error, caused by looseness of the microfluidic chip 100 in the detection apparatus 200, in detection results.

In order to realizing a fixation of the microfluidic chip 100 during detection, embodiments of the present disclosure provide a crimping device 201 for the microfluidic chip 100.

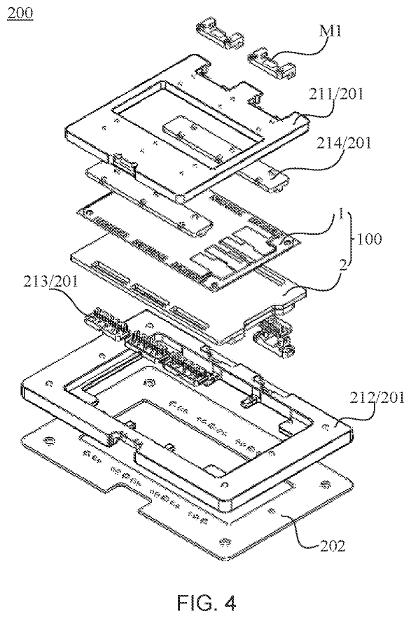

As shown in FIG. 4, the crimping device 201 includes a cover plate 211, a bottom plate 212 and a probe assembly 213.

The bottom plate 212 and the cover plate 211 are assembled with each other. During detection, the microfluidic chip 100 is disposed between the bottom plate 212 and the cover plate 211 for fixation.

For example, it may be possible to only place the first substrate 1 of the microfluidic chip 100 in the crimping device 201 for detection. For example, it may be possible to only place the first substrate 1 provided with the first base 11, the first conductive layer 12, the insulating layer 13 and the second conductive layer 14 in the crimping device 201 for detection. Alternatively, it may be possible to only place the first substrate 1 provided with the first base 11, the first conductive layer 12, the insulating layer 13, the second conductive layer 14, the first hydrophobic layer 15 and the dielectric layer 16 in the crimping device 201 for detection. In this way, it is conductive to determine whether the first substrate 1 have a short-circuit fault or an open-circuit fault after each preparation process, thereby improving the product yield.

Alternatively, it may also be possible to place the microfluidic chip 100 after the first substrate 1 is assembled with the second substrate 2 in the opposing setting to for the cell in the crimping device 201 for detection, so that detection and screening of the finished microfluidic chip 100 are realized, which prevents an inferior product with a short-circuit fault or an open-circuit fault from being provided to a client side.

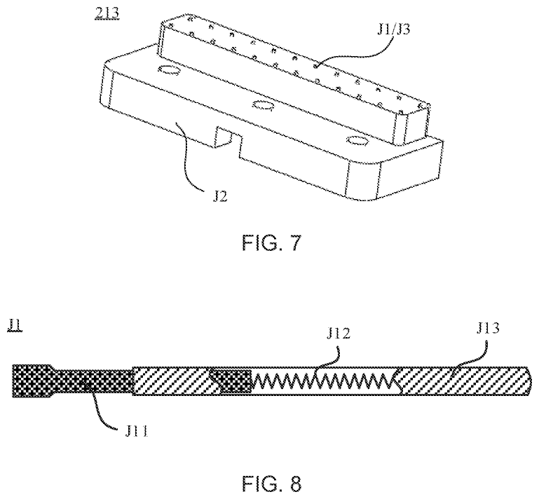

For example, after the microfluidic chip 100 is fixed to the crimping device 201, the second conductive layer 14 of the first substrate 1 is closer to the bottom plate 212 of the crimping device 201 with respect to the first base 11, so that electrodes to be detected of the second conductive layer 14 are connected with the probe assembly 213 (with reference to FIG. 7) in the bottom plate 212.

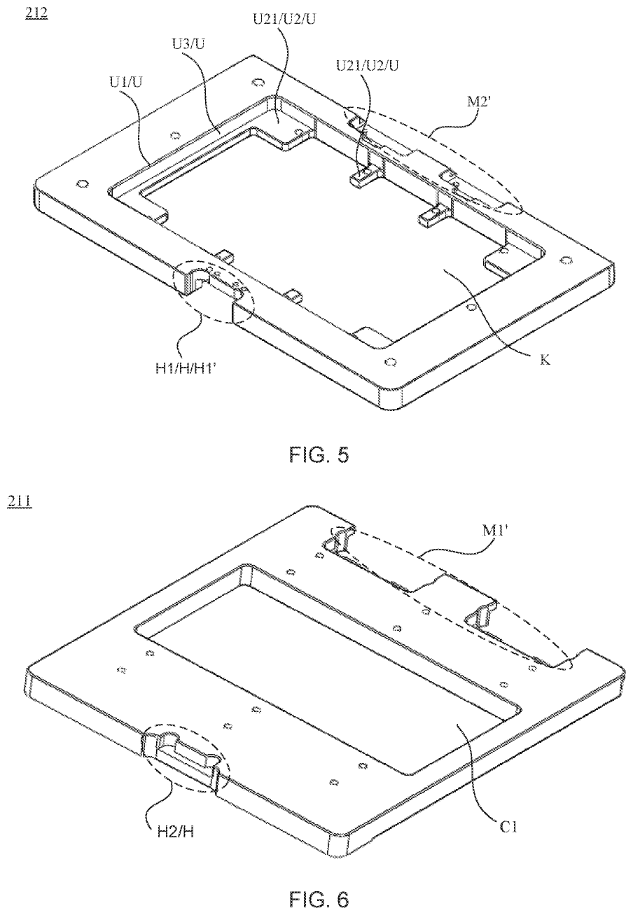

With reference to FIG. 5, the bottom plate 212 is provided with a carrying recess U. A mouth U1 of the carrying recess U faces the cover plate 211. A bottom U2 of the carrying recess U is provided with an opening K.

The carrying recess U is configured to provide a space for placing the microfluidic chip 100. That is, the microfluidic chip 100 is disposed in the carrying recess U during detection.

For example, sizes, such as a length and a width, of the carrying recess U are matched with a length and a width of the microfluidic chip 100, so that the microfluidic chip 100 may be embedded in the carrying recess U. Thus, a movement of the microfluidic chip 100 may be limited by the carrying recess U. This avoids a displacement of the microfluidic chip 100 in a direction parallel to a surface of the bottom U2 in a subsequent detection process, and thus avoids a large error in detection results due to such a displacement.

For example, the bottom U2 may be provided with a plurality of openings K, and the plurality of openings K are sequentially arranged along a wall U3. Alternatively, for example, with reference to FIG. 5, the bottom U2 may be provided with one opening K.

It will be noted that, the opening K is configured to allow probes of the probe assembly 213 to pass through the bottom plate 212. A specific structure of the opening K is not limited in embodiments of the present disclosure.

For example, with reference to FIG. 6, the cover plate 211 includes a first window C1. The first window C1 is configured to expose the microfluidic chip 100 disposed in the carrying recess U of the bottom plate 212 after the cover plate 211 is assembled with the bottom plate 212, so as to observe a crimping state of the microfluidic chip 100. This avoids a crimping position deviation of the microfluidic chip 100, and may improve accuracy of a detection result for the microfluidic chip 100.

With reference to FIG. 7, the probe assembly 213 includes a plurality of probes J1.

Ends, proximate to the cover plate 211, of the plurality of probes J1 are configured to be in contact with the microfluidic chip 100; and ends, away from the cover plate 211, of the plurality of probes J1 passes through the opening K.

For example, the ends, proximate to the cover plate 211, of the plurality of probes J1 are configured to be in contact with electrodes to be detected of the microfluidic chip 100. For example, the plurality of probes J1 are in electrical contact with the plurality of bonding electrodes P of the microfluidic chip 100 in a one-to-one correspondence.

For example, the ends, away from the cover plate 211, of the plurality of probes J1 are in contact with the circuit board 202 after passing through the opening K. During detection, the microfluidic chip 100 is placed on the bottom U2 of the carrying recess U, in contact with the plurality of probes J1, and finally electrically connected with the circuit board 202 through the probes J1. In this way, the detection for the microfluidic chip 100 by the circuit board 202 is realized. For example, a short-circuit detection or an open-circuit detection between the electrodes to be detected of the microfluidic chip 100 is realized.

For example, the probes J1 have elasticity in length directions thereof. After the microfluidic chip 100 is placed in the carrying recess U and in contact with the probes J1, due to an elastic action of the probes J1 and a pressing action of the cover plate 211, it may be ensured that the microfluidic chip 100 is firmly fixed in the crimping device 201. Therefore, it is ensured that the electrodes to be detected of the microfluidic chip 100 are in full contact with the probes J1, which avoids a problem that the accuracy of the detection result is reduced due to looseness of the microfluidic chip 100.

For example, a length of the probes J1 may be in a range of 6 mm to 10 mm, inclusive. For example, the length may be 6 mm, 6.5 mm, 7 mm, 8.425 mm or 10 mm.

For example, an elastic deformation range of the probes J1 may be from 1 mm to 2 mm, inclusive. For example, an amount of compressive deformation or an amount of stretch deformation of the probes J1 may be 1 mm to 2 mm, inclusive. For example, the amount of compressive deformation or the amount of stretch deformation may be 1 mm, 1.25 mm, 1.5 mm, 1.725 mm or 2 mm.

For example, as shown in FIG. 8, a probe J1 includes a conductive shaft J11, an elastic structure J12 and a sleeve J13.

For example, at least a portion of the conductive shaft J11 and the elastic structure J12 are disposed inside the sleeve J13. An end of the elastic structure J12 is fixedly connected with the sleeve J13, and the other end of the elastic structure J12 is fixedly connected with the conductive shaft J11. Due to expansion and contraction of the elastic structure J12, the conductive shaft J11 can be driven to move in the sleeve J13 in an axial direction of the sleeve J13.

An end, away from the sleeve J13, of the conductive shaft J11 is configured to be in contact with the microfluidic chip 100; and an end, away from the conductive shaft J11, of the sleeve J13 is configured to be in contact with the circuit board 202 after passing through the opening K. The elastic structure J12 is configured to provide elasticity for the probe J1, so that firmness of the contact of the probe J1 with the microfluidic chip 100 is enhanced.

For example, the conductive shaft J11, the elastic structure J12 and the sleeve J13 are all made of conductive materials. For example, the conductive materials are metal or alloy materials.

For example, the elastic structure J12 may be a spring.

For example, an elastic deformation range of the elastic structure J12 may be from 1 mm to 2 mm, inclusive. For example, an amount of compressive deformation or an amount of stretch deformation of the elastic structure J12 may be in a range of 1 mm to 2 mm, inclusive. For example, the amount of compressive deformation or the amount of stretch deformation may be 1 mm, 1.25 mm, 1.5 mm, 1.725 mm or 2 mm.

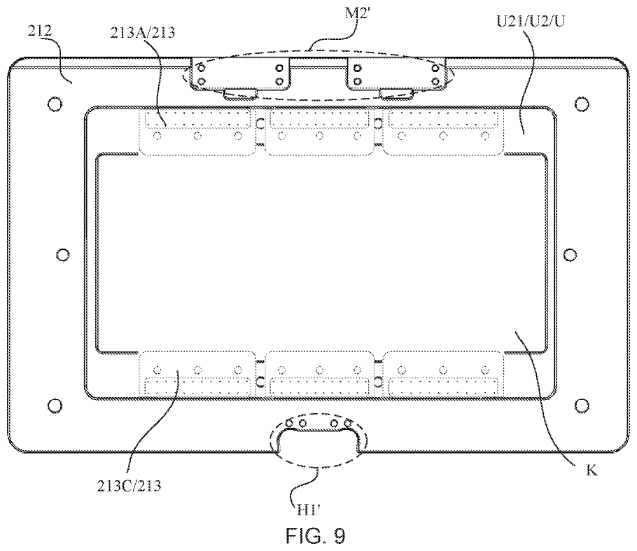

With reference to FIG. 9, the probe assembly 213 is fixedly connected with the bottom U2 of the carrying recess U.

For example, as shown in FIG. 9, the probe assembly 213 corresponds to a position of the opening K, such that the probes J1 of the probe assembly 213 can pass through the opening K.

For example, as shown in FIG. 5, the bottom U2 of the carrying recess U includes a plurality of carrying portions U21. The plurality of carrying portions U21 are arranged at intervals around the wall U3 of the carrying recess U. At least one of the plurality of carrying portions U21 is raised relative to the wall U3.

As shown in FIG. 9, the probe assembly 213 is disposed between two adjacent carrying portions U21, and two ends of the probe assembly 213 are respectively fixedly connected with the two adjacent carrying portions U21. For example, the two ends of the probe assembly 213 are respectively fixedly connected with the two adjacent carrying portions U21 by bolt structures.

By providing the carrying portions U21, the probe assembly 213 may be fixed to the bottom plate 212 while the space for placing the microfluidic chip 100 is provided.

For example, as shown in FIG. 7, the probe assembly 213 further includes a needle module J2. The needle module J2 is fixedly connected with the bottom U2 of the carrying recess U. For example, two ends of the needle module J2 are respectively fixedly connected with the two adjacent carrying portions U21.

With reference to FIG. 7, the needle module J2 is provided with a plurality of mounting holes J3. A probe J1 passes through a respective mounting hole J3, and is fixed in the mounting hole J3.

For example, axis directions of the mounting holes J3 are substantially same as a setting direction of the opening K, so that the probes J1 can pass through the opening K to be in contact with the circuit board 202 while being fixed in the mounting holes J3.

For example, in a case where the probe J1 includes the sleeve J13, the sleeve J13 is fixed to the needle module J2.

For example, an outer wall of the sleeve J13 is provided with a snap-fit portion, a wall of the mounting hole J3 is provided with an annular latching slot matched with the snap-fit portion, and the sleeve J13 can enter an inside of the mounting hole J3 through rotation, and snap to the annular latching slot by means of the snap-fit portion. In this way, the probe J1 is fixed to the needle module J2.

For example, the end, away from the conductive shaft J11, of the sleeve J13 is mounted to the mounting hole J3, passes through the mounting hole J3 to expose at least a portion of the sleeve J13, which facilitates an electrical contact between the sleeve J13 and the circuit board 202 to realize signal transmission.

In the crimping device 201 provided in some embodiments of the present disclosure, by providing the cover plate 211 and the bottom plate 212 which are assembled with each other in the opposing setting to form a cell structure, the space for placing the microfluidic chip 100 is provided, and the microfluidic chip 100 is fixed by the cell structure, which avoids that accuracy of a detection result is reduced due to a displacement of the microfluidic chip 100 during detection.

In addition, by providing the opening K in the bottom U2 of the carrying recess U of the bottom plate 212, providing the probe assembly 213, and fixing the probe assembly 213 in the opening K, the probes J1 are enabled to be in electrical contact with a circuit driving element (such as the circuit board 202) outside the crimping device 201 through the opening K. Therefore, after the microfluidic chip 100 is placed in the carrying recess U of the crimping device 201 and in contact with the probes J1, the microfluidic chip 100 is connected with the circuit driving element outside the crimping device 201, so that a fault detection for the microfluidic chip 100 is realized.

The crimping device 201 provided in the embodiments of the present disclosure has a simple structure, and is convenient to operate, which may effectively improve an efficiency of detection and screening for microfluidic chips 100, and improve the product yield of the microfluidic chips 100.

In the embodiments described above, in a case where an entire microfluidic chip 100 is placed in the crimping device 201, the second substrate 2 of the microfluidic chip 100 is disposed closer to the bottom plate 212 than the first substrate 1, so that the bonding electrodes P of the first substrate 1 is closer to the probe assembly 213 of the crimping device 201 with respect to the first base 11, which facilitates an electrical contact between the probe assembly 213 and the electrodes to be detected of the microfluidic chip 100.

Alternatively, it may be possible to place only the first substrate 1 of the microfluidic chip 100 in the crimping device 201 for detection. In this case, in the first substrate 1, the second conductive layer 14 provided with the bonding electrodes P is disposed closer to the bottom plate 212 with respect to the first base 11, so that the bonding electrodes P of the first substrate 1 are in contact with the probes J1 of the probe assembly 213. Therefore, the detection for the electrodes to be detected of the first substrate 1 is realized.

In some embodiments, as shown in FIG. 1, the crimping device 201 includes a plurality of probe assemblies.

At least one of the plurality of probe assemblies 213 is a bonding probe assembly 213A. Ends, proximate to the cover plate 211, of a plurality of probes J1 of the bonding probe assembly 213A are configured to be in contact with the bonding electrodes P of the microfluidic chip 100, so as to perform a fault detection on circuits corresponding to the bonding electrodes P of the microfluidic chip 100.

The bonding probe assembly 213A is disposed proximate to the wall U3 of the carrying recess U. The bonding electrodes P of the microfluidic chip 100 are disposed on a peripheral region of the microfluidic chip 100. By setting the bonding probe assembly 213A proximate to the wall U3 of the carrying recess U, it is conductive to an electrical contact between the bonding probe assembly 213A and the bonding electrodes P.

For example, after the first substrate 1 is completely formed, for example, after the first hydrophobic layer 15 is formed, or after the first substrate 1 is assembled with the second substrate 2, the driving electrodes Q are covered by a film layer such as the first hydrophobic layer 15, and only the bonding electrodes P are in an exposed state. By providing the bonding probe assembly 213A, setting the probes J1 of the bonding probe assembly 213A in contact with the exposed bonding electrodes P, and detecting conduction states between different bonding electrodes P, short-circuit detections between different circuits in which the different bonding electrodes are located are realized.

For example, the first substrate 1 includes two bonding electrodes P, and the two bonding electrodes P respectively form two circuits (each with reference to a circuit L′ shown in FIG. 24). That is, the two bonding electrodes P are electrically connected with different driving electrodes Q, respectively. Thus, the different driving electrodes Q are controlled dependently of each other. By detecting the two bonding electrodes P, for example, detecting whether the two bonding electrodes P are shorted to each other, it may be possible to determine whether the two circuits corresponding to the two bonding electrodes P are shorted to each other; and if so, different driving electrodes Q respectively electrically connected with the two bonding electrodes P cannot be controlled dependently of each other, which means that the microfluidic chip 100 is at fault.

The crimping device 201 provided in the embodiments realizes fault detection of a plurality of circuits (circuits formed by electrically connecting the driving electrodes Q with the bonding electrodes P) in the microfluidic chip 100 by multiplexing the bonding electrodes P of the microfluidic chip 100 as detection electrodes without changing a structure of the microfluidic chip 100.

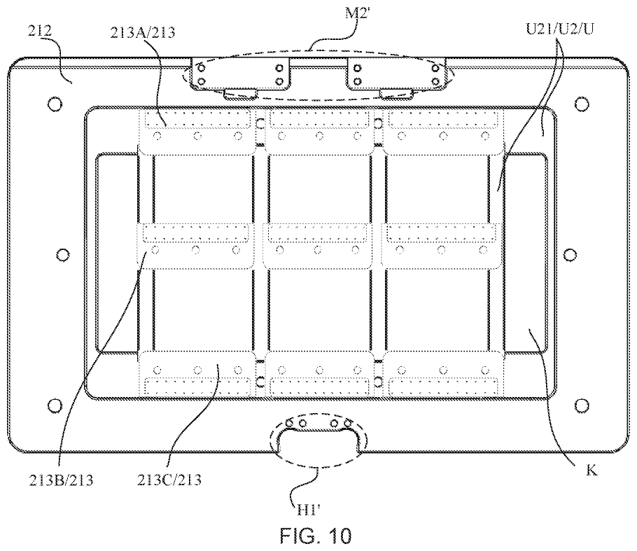

In some embodiments of the present disclosure, as shown in FIGS. 26 and 10, the plurality of probe assemblies 213 further includes at least one driving probe assembly 213B. Ends, proximate to the cover plate 211, of a plurality of probes J1 of the driving probe assembly 213B are configured to be in contact with the driving electrodes Q of the microfluidic chip 100, so as to perform fault detections on circuits corresponding to the driving electrodes Q of the microfluidic chip 100.

With respect to the bonding probe assembly 213A, the driving probe assembly 213B is disposed away from the wall U3 of the carrying recess U. The driving electrodes Q of the microfluidic chip 100 are disposed in a middle portion of the microfluidic chip 100, and the bonding electrodes P are disposed at least partially around the driving electrodes Q. By setting the driving probe assembly 213B away from the wall U3 of the carrying recess U, it is conductive to an electrical contact between the driving probe assembly 213B and the driving electrodes Q.

For example, before the first hydrophobic layer 15 is provided to the first substrate 1, for example, in a case where the first substrate 1 includes only the first base 11, the first conductive layer 12, the insulating layer 13 and the second conductive layer 14, both the driving electrodes Q and the bonding electrodes P located in the second conductive layer 14 are in an exposed state. By providing the bonding probe assembly 213A and the driving probe assembly 213B, the bonding probe assembly 213A may be in contact with the bonding electrodes P, and the driving probe assembly 213B may be in contact with the driving electrodes Q, so that a conduction state or a non-conduction state between any two electrodes (including bonding electrodes P and driving electrodes Q) may be detected.

For example, the first substrate 1 includes one bonding electrode P, and the bonding electrode P is electrically connected with four driving electrodes Q to form a circuit (with reference to the circuit L′ shown in FIG. 24 or a circuit L′ shown in FIG. 25). A conduction state between the bonding electrode P and a driving electrode Q located at an end of the circuit may be detected; and if a detection result of open circuit is displayed, it indicates that the circuit is broken, and the bonding electrode P cannot successfully drive the driving electrodes Q electrically connected therewith. Alternatively, a conduction state between two driving electrodes Q electrically connected with each other may be detected, so that a specific position where an open-circuit or a short-circuit fault occurs may be determined, which is conductive to subsequent troubleshooting.

In the crimping device 201 provided in the embodiments of the present disclosure, by providing the bonding probe assembly 213A and the driving probe assembly 213B, not only short-circuit detections between circuits corresponding to different bonding electrodes P may be realized by detecting the different bonding electrodes P, but also open-circuit detections between a bonding electrode P and a driving electrode Q electrically connected with the bonding electrode P may be realized by detecting the bonding electrode P and the driving electrode Q electrically connected therewith.

In some embodiments, as shown in FIG. 10, at least one of the plurality of probe assemblies 213 is a detection probe assembly 213C.

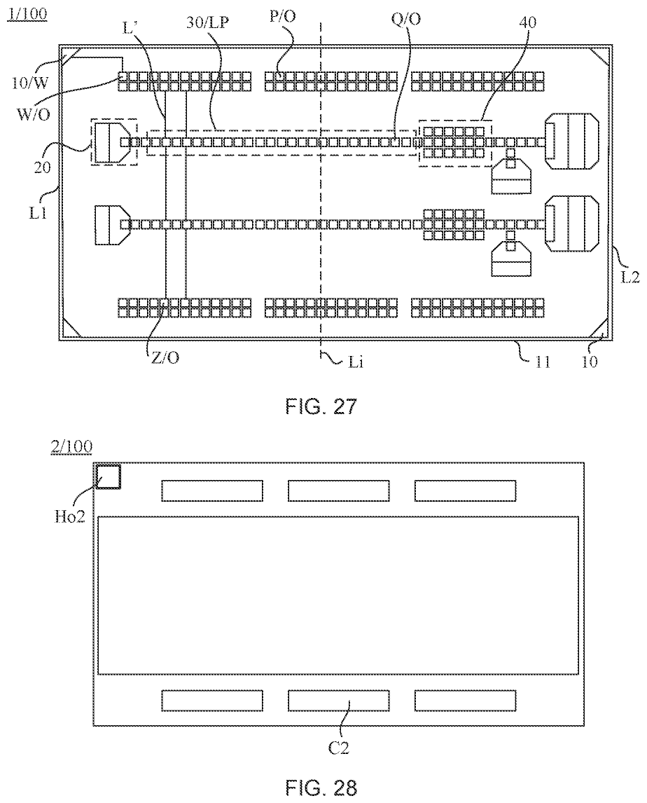

For example, for some microfluidic chips 100, For example, for some microfluidic chips 100, a plurality of detection electrodes Z (with reference to FIG. 27) disposed on the first substrate 1 are further included therein. A circuit where a bonding electrode P is located has a detection electrode Z electrically connected at an end of the circuit away from the bonding electrode P. That is, for a single circuit, a bonding electrode P, at least one driving electrode Q and a detection electrode Z are electrically connected with each other in sequence.

The detection electrodes Z are provided in the second conductive layer 14; and at positions where the detection electrodes Z are located, the first hydrophobic layer 15 and the dielectric layer 16 are hollowed out to expose the detection electrodes Z.

In embodiments where the microfluidic chip 100 is not provided with detection electrodes Z, after the first substrate 1 is formed, for example, after the first hydrophobic layer 15 is formed, or after the first substrate 1 is assembled with the second substrate 2, the driving electrodes Q are covered by a film layer such as the first hydrophobic layer 15, and only the bonding electrodes P is in an exposed state. That is, for a single circuit (with reference to a circuit L′ in FIG. 25), only one electrode can be detected. In this case, it may only be possible to perform short-circuit detections on different circuits.

In embodiments where the microfluidic chip 100 is provided with the detection electrodes Z, the bonding electrodes P and the detection electrodes Z are both in an exposed state. That is, for a single circuit (with reference to a circuit L′ in FIG. 27), two electrodes (one bonding electrode P and one detection electrode Z) that are located at two ends of the circuit may be detected. In this case, not only may short-circuit detections between different circuits be realized, but also an open-circuit detection for a same circuit may be realized.

Ends, proximate to the cover plate 211, of the plurality of probes J1 of the detection probe assembly 213C are configured to be in contact with the detection electrodes Z of the microfluidic chip 100, so that fault detections of circuits corresponding to the detection electrodes Z of the microfluidic chip 100 are realized.

Relative to the driving probe assembly 213B, the probing probe assembly 213C is located proximate to the wall U3 of the carrying recess U. The driving electrodes Q of the microfluidic chip 100 are disposed in the middle portion of the microfluidic chip 100, and the bonding electrodes P and the detection electrodes Z are arranged at least partially around the driving electrodes Q. By setting the detection probe assembly 213C proximate to the wall U3 of the carrying recess U, it is conductive to realizing an electrical contact between the detection probe assembly 213C and the detection electrodes Z.

It will be noted that, the bonding electrodes P may be substantially same as the detection electrodes Z in shape, sizes, etc., and a position of the bonding electrodes P and a position of the detection electrodes Z may be interchanged. Accordingly, a position of the bonding probe assembly 213A and a position of the detection probe assembly 213C may be interchanged.

By providing the bonding probe assembly 213A and the detection probe assembly 213C, after the preparation of the first substrate 1 is completed, for example, after the first hydrophobic layer 15 is formed, or after the first substrate 1 is assembled with the second substrate 2, the crimping device 202 provided in the embodiments of the present disclosure may realize not only short-circuit detections between circuits corresponding to different bonding electrodes P by the detecting different bonding electrodes P, but also an open-circuit detection of a circuit corresponding to a bonding electrode P and a detection electrode Z electrically connected with the bonding electrode P by detecting the bonding electrode P and the detection electrodes Z electrically connected therewith.

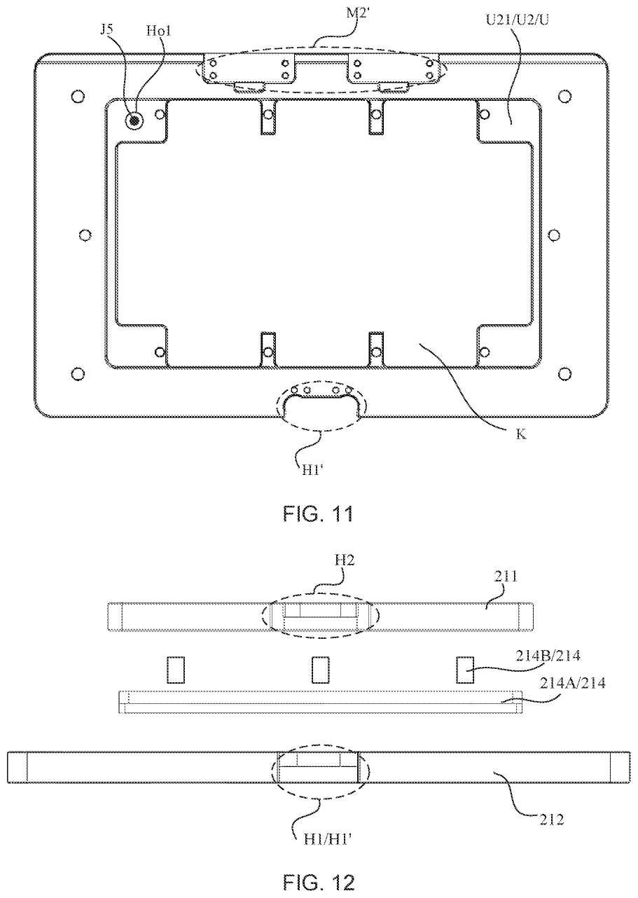

In some embodiments, as shown in FIG. 11, the bottom U2 of the carrying recess U is further provided with a first through hole Ho1. The crimping device 201 further includes a grounding probe J5.

With reference to FIG. 11, the grounding probe J5 is fixedly connected with the bottom U2 of the carrying recess U. For example, the grounding probe J5 includes a sleeve J13, and the grounding probe J5 is fixedly connected with the bottom U2 of the carrying recess U through the sleeve J13.

With reference to FIG. 11, an end, proximate to the cover plate 211, of the grounding probe J5 is configured to be in contact with the microfluidic chip 100, e.g., to be in electrical contact with the grounding electrode 10 of the microfluidic chip 100, so that a fault detection for a circuit corresponding to the grounding electrode 10 of the microfluidic chip 100 is realized.

With reference to FIG. 11, an end, away from the cover plate 211, of the grounding probe J5 passes through the first through hole Ho1. For example, the end, away from the cover plate 211, of the grounding probe J5 is in electrical contact with the circuit board 202 after passing through the through hole Ho1.

By providing the grounding probe J5, setting the end of the grounding probe J5 to be in contact with the microfluidic chip 100, and setting the other end of the grounding probe J5 to be in contact with an electronic element (such as the circuit board 202) outside the crimping device 201, it may be possible to realize a detection for a circuit corresponding to the grounding electrode 10 of the microfluidic chip 100.

In some embodiments, the plurality of probes J1 and the grounding probe J5 have elasticity in respective length extension directions thereof. That is, both the probes J1 and the grounding probe J5 have elasticity, so that contact firmness between the probes J1 and electrodes to be detected corresponding thereto and contact firmness between the grounding probe J5 and an electrode to be detected corresponding thereto may be enhanced by the elasticity, which ensures detection effects.

An elastic deformation range of the grounding probe J5 is greater than an elastic deformation range of at least one of the plurality of probes J1.

For example, a difference value between the elastic deformation range of the grounding probe J5 and the elastic deformation range of the at least one of the plurality of probes J1 is in a range of 1 mm to 3 mm, inclusive. For example, the elastic deformation range of the probe J1 may be from 1 mm to 2 mm, inclusive; and the elastic deformation range of the grounding probe J5 may be from 2 mm to 5 mm, inclusive.

For example, a minimum length of the grounding probe J5 after compression is less than a minimum length of the at least one of the plurality of probes J1 after compression. For example, in a case where the minimum length of the probe J1 after compression is 6 mm, the minimum length of the grounding probe J5 after compression may be 3 mm.

For example, a length of the grounding probe J5 in an absence of a force is substantially equal to a length of the probes J1 in an absence of a force.

For example, the microfluidic chip 100 further includes a conductive foam disposed between the first substrate 1 and the second substrate 2. The grounding electrode 10 located in the first substrate 1 is electrically connected with the common electrode layer 22 of the second substrate 2 through the conductive foam.

In a case where the entire microfluidic chip 100 is placed in the crimping device 201 for detection, the end of the grounding probe J5 proximate to the cover plate 211 is in contact with the conductive foam of the microfluidic chip 100, and an electrical conduction between the grounding probe J5 and the grounding electrode 10 is realized through the conductive foam. In this case, with respect to the contact between the probes J1 and the driving electrodes Q, a thickness of the conductive foam is added between the grounding probe J5 and the grounding electrode 10. That is, the grounding probe J5 is more compressed than the probes J1. By setting the elastic deformation range of the grounding probe J5 to be greater than the elastic deformation range of the at least one of the plurality of probes J1, it is may possible to effectively have the grounding probe J5 applicable to both the case where the entire microfluidic chip 100 is placed in the crimping device 201 and the case where only the first substrate 1 of the microfluidic chip 100 is placed in the crimping device 201.

In some embodiments, as shown in FIG. 4, the crimping device 201 further includes a press-fit structure 214. The press-fit structure 214 is configured to enhance a pressing force of the cover plate 211 on the microfluidic chip 100 placed in the carrying recess U, so that the microfluidic chip 100 is placed firmly. Thus prevents detection result accuracy from being reduced due to looseness of the microfluidic chip 100.

With reference to FIGS. 4 and 12, the press-fit structure 214 is disposed on a side of the cover plate 211 facing the bottom plate 212. The press-fit structure 214 is elastic. For example, the press-fit structure 214 is retractable in a thickness direction of the cover plate 211.

For example, the press-fit structure 214 may be a gasket having elasticity.

For example, as shown in FIG. 12, the press-fit structure 214 includes a press-fit plate 214A and elastic member(s) 214B. The elastic member(s) 214B are located between the press-fit plate 214A and the cover plate 211. An end of an elastic member 214B is connected with the press-fit plate 214A, and the other end of the elastic member 214B is connected with the cover plate 211. The elastic member(s) 214B may be, for example, spring(s).

By setting the pressing structure 214 to have elasticity, a size of the space formed between the bottom plate 212 and the cover plate 211 for placing the microfluidic chip 100 may be changed in the thickness direction of the cover plate 211 according to an elastic deformation amount of the press-fit structure 214. This allows the crimping device 201 to be applicable to microfluidic chips 100 with different thicknesses. In another aspect, since having elasticity, the press-fit structure 214 may provide a pressure in a direction from the microfluidic chip 100 to the bottom plate 212 after the microfluidic chip 100 is placed on the bottom plate 212. Thus, contact firmness between the microfluidic chip 100 and the probes J1 of the probe assembly 213 is further enhanced, which avoids looseness of the microfluidic chip 100 during detection, and then improves detection accuracy of the detection apparatus 200.

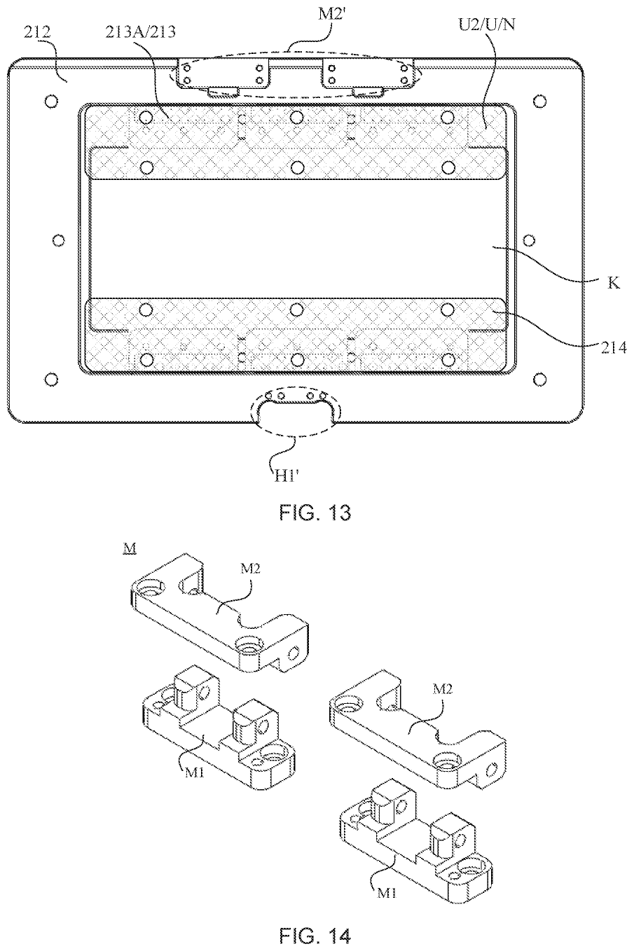

With reference to FIG. 13, in a case where the cover plate 211 is buckled with the bottom plate 212, an orthographic projection of the press-fit structure 214 on a reference plane N at least partially overlaps with orthogonal projections of the probe assemblies 213 on the reference plane N. The reference plane N is a plane where a surface, proximate to the cover plate 211, of the bottom U2 of the carrying recess U is located.

For example, as shown in FIGS. 12 and 13, in the embodiments where the press-fit structure 214 includes the press-fit plate 214A and the elastic member(s) 214B, an orthographic projection of the press-fit plate 214A on the reference plane N at least partially overlaps with the orthographic projections of the probe assemblies 213 on the reference plane N in the case where the cover plate 211 is buckled with the bottom plate 212.

The orthographic projections of the press-fit structure 214 and the probe assemblies 213 on the reference plane N are set to be at least partially overlapped with each other. That is, the press-fit structure 214 provides a pressure at positions, corresponding to the probe assemblies 213, of the microfluidic chip 100. This may ensure that the electrodes to be detected of the microfluidic chip 100 are in firm contact with the probes J1 of the probe assemblies 213, which avoids looseness of the microfluidic chip 100 during detection, and improves the detection accuracy of the detection apparatus 200.

For example, the press-fit plate 214A may be parallel to the reference plane N.

For example, an elastic expansion and contraction direction of the elastic element 214B may be perpendicular to the reference plane N, so as to provide a pressure perpendicular to the reference plane N for the microfluidic chip 100. This ensures that the electrodes to be detected of the microfluidic chip 100 are in firm contact with the probes J1 of the probe assembly 213, which avoids looseness of the microfluidic chip 100 during detection, and improves the detection accuracy of the detection apparatus 200.

In some embodiments, as shown in FIG. 14, the crimping device 201 further includes hinge structures M.

With reference to FIG. 14, a hinge structure M includes a first hinge M1 and a second hinge M2 movably connected with each other. The first hinge M1 is fixedly connected with the cover plate 211, and the second hinge M2 is fixedly connected with the bottom plate 212. The cover plate 211 of the crimping device 201 may be assembled with the bottom plate 212 of the crimping device 201 by means of the hinge structures M.

For example, the cover plate 211 of the crimping device 201 may also be assembled with the bottom plate 212 of the crimping device 201 by means of a gemel structure.

For example, first hinges M1 are disposed at a hinge mounting location M1′ of the cover plate 211 as shown in FIG. 6, and second hinges M2 are disposed at a hinge mounting location M2′ of the bottom plate 212 as shown in FIG. 5.

In some embodiments, as shown in FIGS. 5 and 6, the crimping device 201 further includes a snap-fit structure H.

The snap-fit structure H includes a buckle H1 and a latching slot H2. The buckle H1 is fixedly connected with one of the cover plate 211 and the bottom plate 212. The latching slot H2 is disposed on the other of the cover plate 211 and the bottom plate 212. The buckle H1 is capable of being snap-fitted to the latching slot H2. The cover plate 211 and the bottom plate 212 of the crimping device 201 may be assembled with each other through the snap-fit structure H.

For example, the buckle H1 is disposed at a snap-fit mounting location H1′ of the cover plate 211 as shown in FIG. 5, and the latching slot H2 is provided in the bottom plate 212 as shown in FIG. 6.

In some embodiments, the crimping device 201 includes the hinge structures M and the snap-fit structure H. With reference to FIGS. 5 and 6, the hinge structures M and the snap-fit structure H are respectively disposed on opposite sides of the cover plate 211 and the bottom plate 212.

By providing the hinge structures M and the snap-fit structure H, and setting the hinge structures M and the snap-fit structure H respectively locate the opposite sides of the cover plate 211 and the bottom plate 212, the cover plate 211 and the bottom plate 212 of the crimping device 201 may be firmly assembled with each other. Therefore, it is ensured that the microfluidic chip 100 is fasten in the carrying recess U, which further avoids looseness of the microfluidic chip 100 during detection, and improves the detection accuracy of the detection apparatus 200.

The circuit board 202 of the detection apparatus 200 is configured to cooperate with the crimping device 201 to detect voltages of the microfluidic chip 100 fixed by the crimping device 201.

In order to realize the detection for the microfluidic chip 100, embodiments of the present disclosure provides a circuit board 202.

With reference to FIG. 4, the circuit board 202 is disposed on a side of the bottom plate 212 of the crimping device 201 away from the cover plate 211.

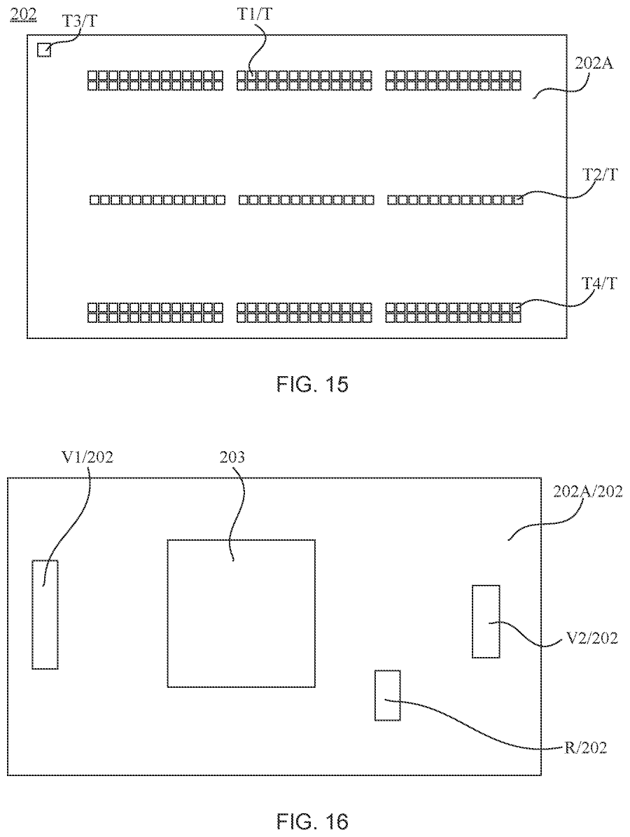

With reference to FIG. 15, the circuit board 202 includes a substrate 202A, and a plurality of pads T disposed on the substrate 202A. A pad T is in contact with a respective probe J1 of the crimping device 201. Through the pads T, detection signals from the circuit board 202 are transmitted to the microfluidic chip 100 fixed by the crimping device 201, so that the detection for the microfluidic chip 100 is realized.

For example, as shown in FIG. 15, the plurality of pads T includes pin pads T1. The pin pads T1 are configured to be in contact with the probes J1 of the bonding probe assembly 213A of the crimping device 201, so that the circuit board 202 is electrically connected with the bonding electrodes P of the microfluidic chip 100, and fault detections (e.g., short-circuit detections or open-circuit detections) of circuits corresponding to the bonding electrodes P of the microfluidic chip 100 are realized.

For example, as shown in FIG. 15, the plurality of pads T may further include driving pads T2. The driving pads T2 are configured to be in contact with the probes J1 of the driving probe assembly 213B of the crimping device 201, so that the circuit board 202 is electrically connected with the driving electrodes Q of the microfluidic chip 100, and fault detections (e.g., open-circuit detections) of circuits corresponding to the driving electrodes Q of the microfluidic chip 100 are realized.

For example, as shown in FIG. 15, the plurality of pads T may further include detection pads T4. The detection pads T4 are configured to be in contact with the probes J1 of the detection probe assembly 213C of the crimping device 201, so that the circuit board 202 is electrically connected with the detection electrodes Z of the microfluidic chip 100, and fault detections (e.g., open-circuit detections) of circuits corresponding to the detection electrodes Z of the microfluidic chip 100 are realized.

For example, corresponding to the electrodes of the microfluidic chip 100, among the plurality of pads T of the circuit board 202, the driving pads T2 are located in a middle region of the circuit board 202, and the pin pads T1 and/or the detection pads T4 are at least partially disposed around the driving pads T2. For example, with reference to FIG. 15, a plurality of pin pads T1 and/or a plurality of detection pads T4 are respectively disposed on two sides of a plurality of driving pads T2.

It will be noted that, the plurality of pads T of the circuit board 202 may include only the pin pads T1, or include both the detection pads T4 and the pin pads T1. Positions of the detection pads T4 and positions of the pin pads T1 are respectively set to correspond to positions of the detection electrodes Z and positions of the bonding electrodes P of the microfluidic chip 100. The positions of the detection pads T4 and the positions of the pin pads T1 may be interchanged. FIG. 15 is only an exemplary illustration, and embodiments of the present disclosure are not limited thereto.

In some embodiments, as shown in FIG. 15, in a case where the crimp device 201 includes the grounding probe J5, the circuit board 202 further includes a grounding pad T3. The grounding pad T3 is in contact with the grounding probe J5. Thereby, the circuit board 202 is electrically connected with the grounding electrode 10 of the microfluidic chip 100, and a fault detection for a circuit corresponding to the grounding electrode 10 of the microfluidic chip 100 is realized.

In some embodiments, as shown in FIG. 16, the circuit board 202 further includes a resistor R. The resistor R is connected in parallel with the processor 203. The resistor R is configured to adjust an overall resistance value of the detection apparatus 200, so that the detection apparatus 200 may be applicable to detections for microfluidic chips 100 with different resistance values.

For example, a resistance value of the resistor R is less than or equal to a resistance value of the processor 203. By setting the resistor R to be connected in parallel with the processor 203 and setting the resistance value of the resistor R less than or equal to the resistance value of the processor 203, a voltage division of the detection apparatus 200 is made to depend on the resistor R with a relatively small resistance. Thereby, an adjustment effect of the resistor R on the overall resistance and the voltage division of the detection apparatus 200 is realized.

For example, the resistance value of the resistor R may be greater than or equal to 33.3 kΩ, and the resistance value of the processor may be 40 kΩ.

In some embodiments, as shown in FIG. 10, the circuit board 202 further includes an industrial personal computer interface V1. The industrial personal computer interface V1 is configured to be electrically connected with the industrial personal computer 300 of the detection system 1000.

In some embodiments, as shown in FIG. 16, the circuit board 202 further includes a power supply interface V2. The power supply interface V2 is configured to be electrically connected with the power supply 400 of the detection system 1000, so as to supply power to the detection apparatus 200 and the industrial personal computer 300.

In some embodiments, the circuit board 202 further includes a detection circuit. The detection circuit is provided therein with a plurality of detection signal lines. The pads T, the processor 203, the resistor R and other components are electrically connected with each other through the plurality of detection signal lines, so as to realize an operation of the detection system 1000.

The processor 203 of the detection apparatus 200 is configured to cooperate with the crimping device 201 and the circuit board 202 to realize the detection for the microfluidic chip 100 fixed by the crimping device 201.

In order to realize the detection for the microfluidic chip 100, embodiments of the present disclosure provides a processor 203.

In some embodiments, as shown in FIG. 16, the processor 203 is disposed on the circuit board 202 and electrically connected with the plurality of pads T.

For example, the plurality of pads T are disposed on a side of the substrate 202A proximate to the bottom plate 212, and the processor 203 is disposed on a side of the substrate 202A away from the bottom plate 212, which facilitates an contact of the pads of the circuit board 202 with the probes J1 of the crimping device 201.

The processor 203 is configured to transmit detection signals to the probes J1 in contact with the pads T through the pads T, receive feedback signals from the probes J1, and process the feedback signals.

Based on the detection system 1000, some embodiments of the present disclosure provide a method for detecting the microfluidic chip 100. The method uses the detection apparatus 200 as described in any one of the above embodiments to detect the microfluidic chip 100.

As shown in FIG. 1, the method includes the following steps.

In a step S1, the microfluidic chip 100 is placed in the carrying recess U of the bottom plate 212 of the detection apparatus 200.

With reference to FIG. 25, the microfluidic chip 100 includes the plurality of electrodes O. An electrode O is in contact with a respective probe J1 of the detection apparatus 200.

For example, with reference to FIG. 25, the plurality of electrodes O may include the plurality of driving electrodes Q and the plurality of bonding electrodes P. When short-circuit detections between different circuits L′ corresponding to different bonding electrodes P are performed, the different bonding electrodes P are in contact with different probes J1 of the detection apparatus 200. When a detecting open-circuit detection within a single circuit L′ corresponding to a bonding electrode P is performed, the bonding electrode P and a driving electrode Q electrically connected with the bonding electrode P are in contact with two probes J1 of the detection apparatus 200, respectively.

For example, with reference to FIG. 27, the plurality of electrodes O may further include the plurality of detection electrodes Z. When a detecting open-circuit detection within a single circuit L′ corresponding to a bonding electrode P is performed, the bonding electrode P and a detection electrode Z electrically connected with the bonding electrode P are in contact with two probes J1 of the detection apparatus 200, respectively.

For example, it may be possible to place only the first substrate 1 of the microfluidic chip 100 in the carrying recess U. Alternatively, it may be possible to place the entire microfluidic chip 100 in the carrying recess U after assembly is completed.

For example, a film layer (e.g., the second conductive layer 14) in which the electrodes O of the microfluidic chip 100 are located is disposed closer to the bottom plate 212 with respect to the first base 11, so that the electrodes O are in contact with the probes J1.

In a step S2, a short-circuit detection between two electrodes O, insulated from each other, of the plurality of electrodes O is performed.

For example, with reference to FIG. 25, different driving electrodes Q are respectively connected with different bonding electrodes P to form two different circuits L′ insulated from each other. In a process of manufacturing the microfluidic chip 100, a short-circuit fault may easily occur between two different circuits L′ insulated from each other. By detecting whether two electrodes O, insulated from each other, of the plurality of electrodes O are shorted to each other, for example, by detecting whether two bonding electrodes O, insulated from each other, of the plurality of bonding electrodes O are shorted to each other, it may be possible to effectively screen out circuit(s) L′ with short-circuit faulty, which improves the product yield of microfluidic chips 100.

In a step S3, an open-circuit detection is performed on a circuit L′.

With reference to FIG. 27, the microfluidic chip 100 includes a plurality of circuits L′. Each circuit L′ includes at least two, connected in series with each other, of the plurality of electrodes O.

In the process of manufacturing the microfluidic chip 100, an open circuit is easily caused between different electrodes O which should be located in a same circuit L′. For example, a bonding electrode P is disconnected with a driving electrode Q which should be electrically connected therewith. Consequently, some driving electrode(s) Q of the microfluidic chip 100 are subject to a transmission failure in a driving process. By detecting whether a circuit L′ is broken, for example, detecting whether a bonding electrode P and a driving electrode Q in a same circuit L′ are disconnected with each other, or detecting whether a bonding electrode P and a detecting electrode Z in a same circuit L′ are disconnected with each other, circuit(s) L′ with an open-circuit fault may be effectively screened out, which improves the product yield of microfluidic chips 100.

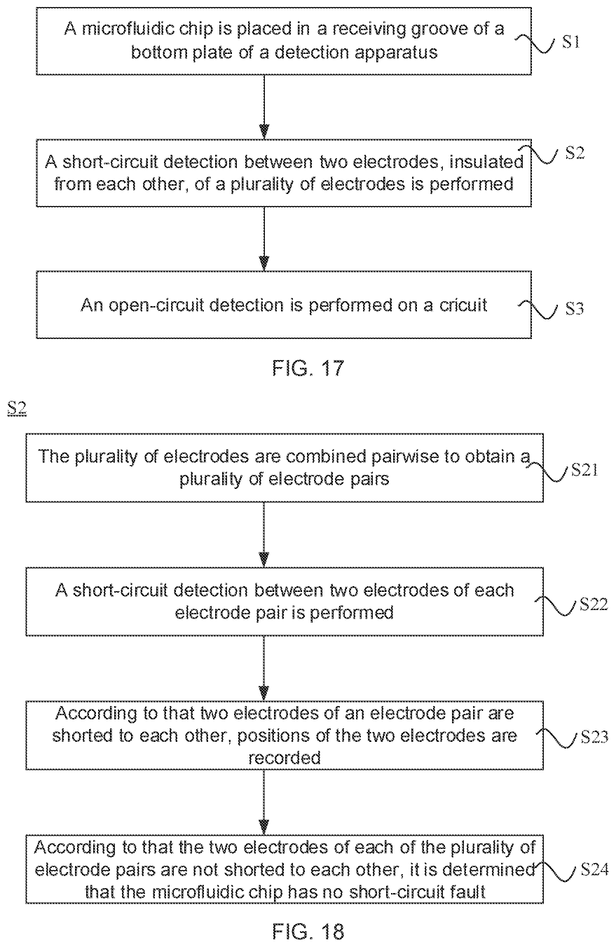

It will be noted that, a sequence of the steps S2 and S3 may be interchanged. FIG. 17 corresponding to the embodiments of the present disclosure only illustrates an example where the step S2 is before the step S3, which does not form a limitation on the sequence of the steps S2 and S3.

It will be further noted that, it may be possible to perform only one of the steps S2 and S3, or it may be possible to perform both of the steps S2 and S3. That is, it may be possible to perform only short-circuit detections, only open-circuit detections, or both the short-circuit detections and the open-circuit detections.

In some embodiments, as shown in FIG. 18, the step that the short-circuit detection between the two electrodes O, insulated from each other, of the plurality of electrodes O is performed includes the following steps.

In a step S21, the plurality of electrodes O are combined pairwise to obtain a plurality of electrode pairs O′ (with reference to FIG. 24).

It will be noted that, when short-circuit detections are performed, the plurality of electrodes O may be all bonding electrodes P. That is, a plurality of bonding electrodes P are combined pairwise.

Two electrodes O of each electrode pair O′ are insulated from each other; and for any two electrode pairs O′, two electrodes of one electrode pair O′ are not completely same as two electrodes of another electrode pair O′. For example, with reference to FIG. 25, each bonding electrode P corresponds to a respective circuit L′, and bonding electrodes P corresponding to different circuits L′ are theoretically insulated from each other.

In a step S22, a short-circuit detection between the two electrodes O of each electrode pair O′ is performed.

For example, the short-circuit detection between the two electrodes O of each electrode pair O′ is equivalent to a short-circuit detection between two different circuits L′ corresponding to the two bonding electrodes P of each electrode pair O′.

In a step S23, according to that two electrodes O of an electrode pair O′ are shorted to each other, positions of the two electrodes O are recorded.

According to that two electrodes O of the electrode pair O′ are shorted to each other, it indicates that two bonding electrodes P of the electrode pair O′ are shorted to each other. That is, two different circuits L′ corresponding to the two bonding electrodes P of the electrode pair O′ are shorted to each other.

Recording the positions of the two electrodes O means recording positions of the two corresponding bonding electrodes P when a short-circuit fault occurs, which facilitates subsequent troubleshooting of the circuits L′ corresponding to the bonding electrodes P with the short-circuit fault.

In a step S24, according to that the two electrodes O of each of the plurality of electrode pairs O′ are not shorted to each other, it is determined that the microfluidic chip 100 has no short-circuit fault.

After all electrode pairs O′ are detected, if the two electrodes O of each the electrode pairs O′ are not shorted to each other, it indicates that there is no short circuit between any two of all circuits L′.

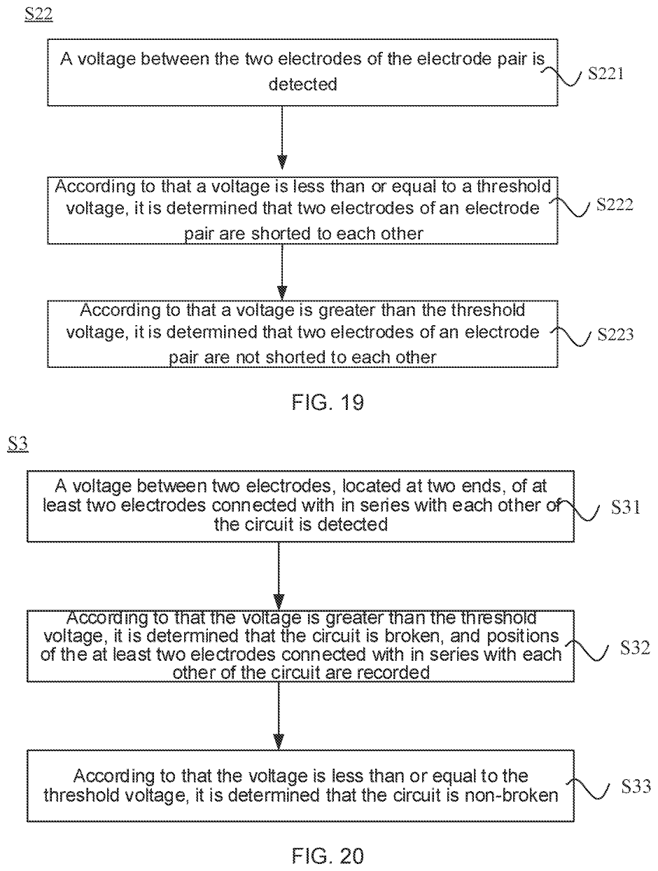

In some embodiments, as shown in FIG. 19, the step that the short-circuit detection between the two electrodes O of each electrode pair O′ is performed includes the following steps.

In a step S221, a voltage between the two electrodes O of the electrode pair O′ is detected.

In a step S222, according to that a voltage is less than or equal to a threshold voltage, it is determined that two electrodes O of an electrode pair O′ are shorted to each other.

In a step S223, according to that a voltage is greater than the threshold voltage, it is determined that two electrodes O of an electrode pair O′ are not shorted to each other.

In some embodiments, as shown in FIG. 20, the step that the open-circuit detection is performed on the circuit L′ includes the following steps.

In a step S31, a voltage between two electrodes O, located at two ends, of at least two electrodes O connected in series with each other of the circuit L′ is detected.

In a step S32, according to that the voltage is greater than the threshold voltage, it is determined that the circuit L′ is broken, and positions of the at least two electrodes O connected in series with each other of the circuit L′ are recorded.

In a step S33, according to that the voltage is less than or equal to the threshold voltage, it is determined that the circuit L′ is non-broken.

In some embodiments, the detection apparatus 200 includes the processor 203 and the resistor R connected in parallel with the processor 203.

The threshold voltage is:

$${V}_{1} = \frac{{R}_{1} V}{{R}_{1} + \frac{{R}_{2} {R}_{3}}{{R}_{2} + {R}_{3}}} ,$$

where V₁ is the threshold voltage, V is a power supply voltage of the detection apparatus 200, R₁ is a resistance between two electrodes of an electrode pair O′ in a case where the two electrodes O are shorted to each other, R₂ is a resistance of the processor 203, and R₃ is a resistance of the resistor R.

In some embodiments, with reference to FIG. 24, the plurality of electrodes O includes two fool-proof electrodes W. The two fool-proof electrodes W are electrically connected with each other, and are asymmetrically arranged with respect to a setting center line L1 of the microfluidic chip 100. The microfluidic chip 100 includes a first side edge L1 and a second side edge L2 that are opposite to each other. The center line L1 is a center line, parallel to the first side edge L1 and the second side edge L2, of the microfluidic chip 100.

As shown in FIG. 6, the method further includes the following steps.

In a step S01, a short-circuit detection between two electrodes O at target positions is performed.

The target positions are positions where the two fool-proof electrodes W are located in a case where the microfluidic chip 100 is placed in the detection apparatus 200 in a correct position.

In a step S02, according to that the two electrodes O at the target positions are not shorted to each other, it is determined that the two electrodes O at the target positions are not the two fool-proof electrodes W, and positions of the first side edge L1 and the second side edge L2 of the microfluidic chip 100 are reversed.

In a step S03, according to that the two electrodes at the target positions are shorted to each other, it is determined that the two electrodes O at the target positions are the two fool-proof electrodes W, and the microfluidic chip 100 is placed in the correct position.

It will be noted that, the steps S01, S02 and S03 are performed before the step S1. That is, before a fault detection for the microfluidic chip 100, a fool-proof detection is performed first, so as to prevent positions are reversed during placing the microfluidic chip on the bottom plate 212, and then avoid a wrong detection result or a failure contact between the electrodes O and the probes J1 due to such a position reverse.

As shown in FIG. 5, some other embodiments of the present disclosure provide a method for manufacturing the microfluidic chip 100. The method includes the following steps.

In a step K1, the first substrate 1 and the second substrate 2 are formed.

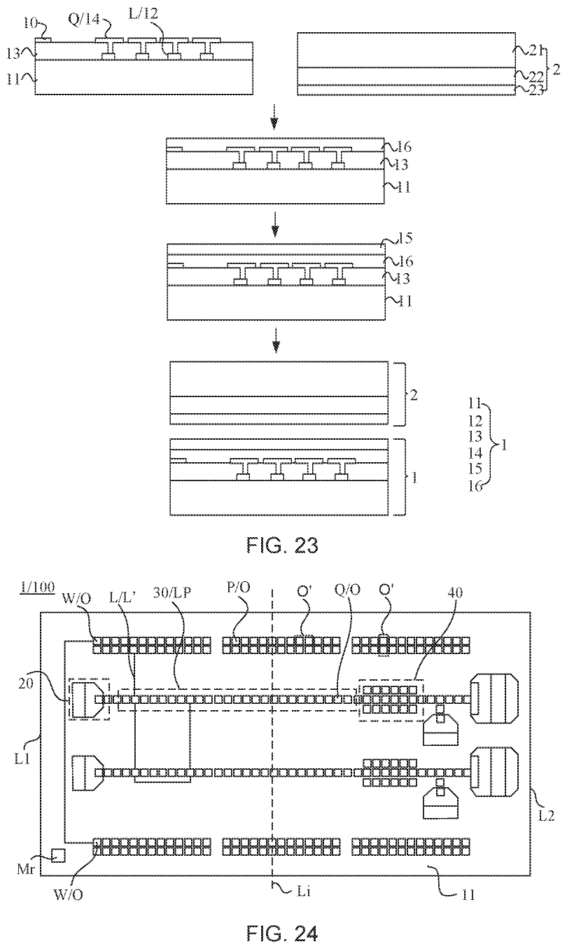

The first substrate 1 includes the plurality of electrodes O. For example, with reference to FIG. 23, the first substrate 1 includes the plurality of driving electrodes Q.

For example, with reference to FIG. 23, a step of forming the first substrate 1 includes sequentially forming the first conductive layer 12, the insulating layer 13 and the second conductive layer 14 on the first base 11.

For example, with reference to FIG. 23, a step of forming the second substrate 2 includes sequentially forming the common electrode layer 22 and the second hydrophobic layer 23 on the second base 21.

In a step K2, the first substrate 1 is placed in the detection apparatus 200 as described in any one of the above embodiments, and the first substrate 1 is detected by the detection method as described in any one of the above embodiments.

In a step K3, according to a detection result that no fault exists, the dielectric layer 16 is formed on the plurality of electrodes O of the first substrate 1.

For example, with reference to FIG. 23, the dielectric layer 16 is disposed on a side of the second conductive layer 14 away from the first base 11.

In a step K4, the first substrate 1 on which the dielectric layer 16 has been formed is detected.

In a step K5, according to a detection result that no fault exists, the first hydrophobic layer 15 is formed on the dielectric layer 16.

For example, with reference to FIG. 23, the first hydrophobic layer 15 is disposed on a side of the dielectric layer 16 away from the first base 11.

In a step K6, the first substrate 1 on which the first hydrophobic layer 15 has been formed is detected.

In a step K7, according to a detection result that no fault exists, the first substrate 1 on which the first hydrophobic layer 15 has been formed is assembled with the second substrate 2.

For example, with reference to FIG. 23, after being assembled with each other, the first substrate 1 and the second substrate 2 have a gap therebetween, and the first hydrophobic layer 15 and the second hydrophobic layer 23 are opposite to each other, so that a channel through which a droplet is allowed to pass is formed.

For example, during each detection for the first substrate 1, the second substrate 2 may be temporarily assembled with the first substrate 1, so as to have the second substrate 2 play a role in supporting the first substrate 1, which avoids looseness of the first substrate 1 in the detection apparatus 200, and thus improves accuracy of detection results.

In conjunction with the detection apparatus 200 and the detection method in the above embodiments, by performing short-circuit and open-circuit detections on the microfluidic chip 100 in a manufacturing process of the microfluidic chip 100, it may be possible to screen out electrodes O with fault in the manufacturing process of the microfluidic chip 100, particularly in a process of forming the first substrate 1, and a next preparation process is performed until no fault occurs. Therefore, it may be possible to reduce a number of re-workings in the manufacturing process of microfluidic chips 100, and effectively improve the product yield of microfluidic chips 100.

In another aspect of embodiments of the present disclosure, a microfluidic chip 100 that may be applicable to the detection apparatus 200 is provided.

With reference to FIG. 1, the microfluidic chip 100 includes the first substrate 1 and the second substrate 2. The second substrate 1 is assembled with the first substrate 2.

As shown in FIGS. 24, 25 and 27, the first substrate 1 includes the two fool-proof electrodes W. The two fool-proof electrodes W are electrically connected with each other, and the two fool-proof electrodes W are arranged asymmetrically with respect to the setting center line L1 of the microfluidic chip 100.

The setting center line L1 passes through midpoints of lines connecting the first side edge L1 and the second side edge L2 at arbitrary positions, and is parallel to the first side edge L1 and the second side edge L2. That is, the first side edge L1 and the second side edge L2 are symmetrically disposed with the setting center line L1 as an axis of symmetry.

For example, the description “the two fool-proof electrodes W are arranged asymmetrically with respect to the setting center line L1 of the microfluidic chip 100” includes the following cases. The two fool-proof electrodes W are respectively disposed at a side of the setting center line L1 proximate to the first side edge L1 and a side of the setting center line L1 away from the first side edge L1, while the two fool-proof electrodes W are asymmetrically arranged with respect to the setting center line L1. Alternatively, the two fool-proof electrodes W are both disposed on a same side, e.g., the side of the setting center line L1 proximate to the first side edge L1 or the side of the setting center line L1 away from the first side edge L1, while the two fool-proof electrodes W are incapable of being symmetrically arranged along the setting center line L1.

By providing the foolproof electrodes W, and setting the two foolproof electrodes W to be asymmetrically arranged with respect to the setting center line L1 of the microfluidic chip 100, it may be possible to determine whether positions of the first side edge L1 and the second side edge L2 of the microfluidic chip 100 are reversed according to a foolproof detection result for the two foolproof electrodes W by the detection apparatus 200. Thus, an inaccurate short-circuit detection result or an inaccurate open-circuit detection result due to a reversed position of the microfluidic chip 100 is avoided.

In some embodiments, as shown in FIGS. 24, 25 and 27, the substrate 1 includes the plurality of driving electrodes Q and the plurality of bonding electrodes P.

A bonding electrode P is electrically connected with at least one driving electrode Q. For example, with reference to FIG. 24, a bonding electrode P is connected in series with four driving electrodes Q to form a circuit L′. Alternatively, for example, with reference to FIG. 25, four driving electrodes Q connected in series pairwise are connected with a same bonding electrode P through two signal lines L. In this case, similarly, the four driving electrodes Q and the bonding electrode P form a single circuit L′.

By detecting a conduction state between different bonding electrodes P, it may be possible to determine whether circuits L′ corresponding to the different bonding electrodes P are shorted to each other.

In some embodiments, as shown in FIG. 27, the first substrate 1 further includes the plurality of detection electrodes Z.

In this case, a single circuit L′ includes one bonding electrode P, at least one driving electrode Q and one detection electrode Z that are electrically connected in sequence.

During an open-circuit detection, by only detecting a conduction state between the bonding electrode P and the detection electrode Z, it may be possible to determine whether an open circuit exists in the circuit L′ corresponding to the bonding electrode P and the detection electrode Z, while there is no need to detect the driving electrode Q, which reduces detection difficulty; and in addition, an open-circuit detection may still be realized in a case where the first substrate 1 is assembled with the second substrate 2, which realizes compatibility of short-circuit detections and open-circuit detections.

In some embodiments, with reference to FIG. 24, the first substrate 1 includes the plurality of driving electrodes Q and the plurality of bonding electrodes P. A bonding electrode P is electrically connected with at least one driving electrode Q.

Two bonding electrodes P of the plurality of bonding electrodes P serves as the two fool-proof electrodes W. The two fool-proof electrodes W are both disposed on a side of the setting center line L1 proximate to the first side edge L1, and the two fool-proof electrodes W are electrically connected with each other.

By multiplexing any two bonding electrodes P as the fool-proof electrodes W, the fool-proof detection is realized, which saves a design space for forming dedicated fool-proof electrodes W.

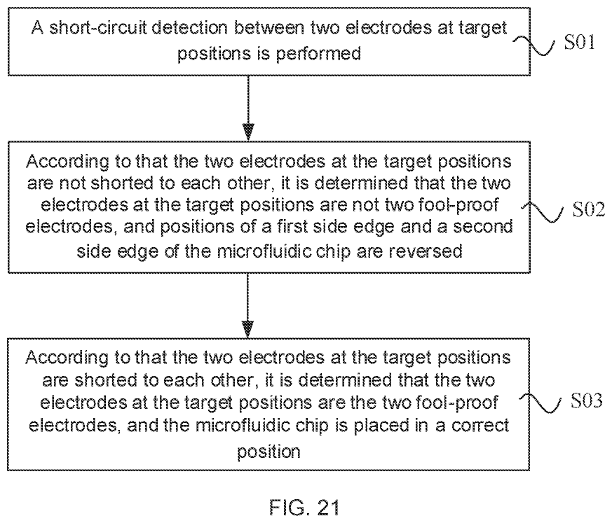

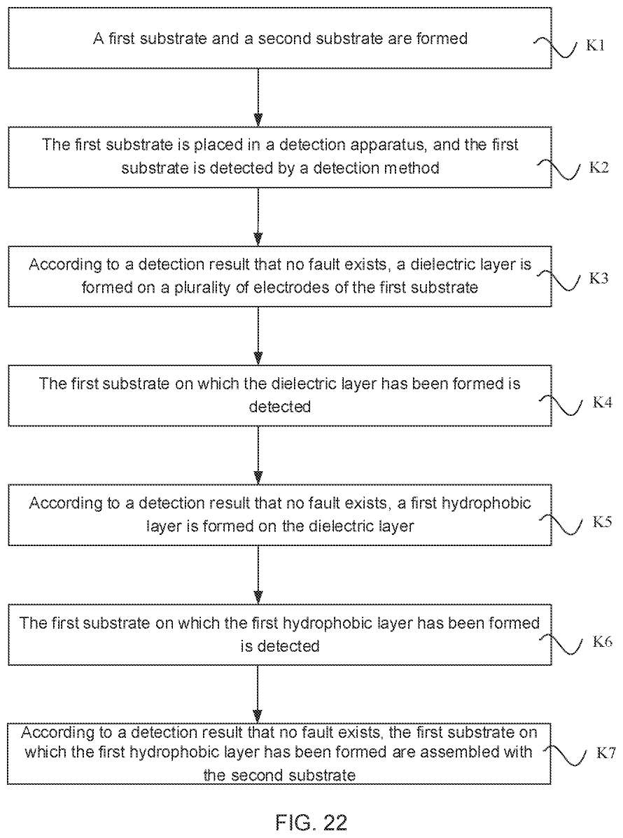

In some embodiments, as shown in FIGS. 4 to 24, the first substrate 1 further includes a fool-proof pattern Mr. The fool-proof pattern Mr is a mark disposed away from a position where the setting center line L1 is located. The fool-proof pattern Mr is configured to prevent the positions of the first side edge L1 and the second side edge L2 of the microfluidic chip 100 from being reversed.

During manufacturing of the microfluidic chip 100, an electrode pattern formed by the driving electrodes Q of the second conductive layer 14 of the first substrate 1 and performances of the first substrate 1 determine a type and accuracy of a process which may be implemented by the microfluidic chip 100.

As shown in FIGS. 24, 25 and 27, the first substrate 1 has a storage region 20 and a transport region 30.

The storage region 20 is provided with storage electrodes. The storage electrodes have a large area, and are used for storing a sample to be tested. The sample to be tested in the storage region 20 forms a droplet 3 due to a step-by-step traction of different storage electrode voltages, and the droplet 3 is drawn to the driving electrodes Q for operation. For example, the storage region 20 is provided with three 3 mm.times.1 mm (3 mm×1 mm) rectangular storage electrodes.

The transport region 30 is provided with a plurality of driving electrodes Q arranged in an array depending on a required electrode pattern. Depending on different required functions of the microfluidic chip 100, an electrode pattern formed by the plurality of driving electrodes Q in the transport region 30, and a number and sizes of the driving electrodes Q may vary.

For example, as shown in FIGS. 24, 25 and 27, the microfluidic chip 100 may include at least one flow channel LP. For example, the microfluidic chip 100 may include four flow channels LP.

For example, with reference to FIG. 24, each flow channel of a plurality of flow channels LP extends in a same direction. Alternatively, for example, a flow channel LP may have a curved shape or an irregular shape, which is not limited in embodiments of the present disclosure.

In some embodiments, as shown in FIGS. 24, 25 and 27, the first substrate 1 has a reaction region 40.

The reaction region 40 is configured such that different types of droplets 3 undergo chemical reactions or physical reactions when being transported to the reaction region 40.

In some embodiments, with reference to FIGS. 25 and 26, the first substrate 1 further includes a temperature sensor F. The temperature sensor F is disposed adjacent to and insulated from at least one driving electrode Q.

For example, the temperature sensor F is disposed in the reaction region 40 and adjacent to at least one driving electrode Q in the reaction region 40. An ambient temperature of the reaction zone 40 is monitored by the temperature sensor F, which facilitates analysis of the droplets 3 in the reaction region 40.

With reference to FIGS. 25 and 26, the temperature sensor F is electrically connected with the two fool-proof electrodes W. That is, electrodes connected with the temperature sensor F is multiplexed as the fool-proof electrodes W to realize the fool-proof detection, which saves a design space for forming dedicated fool-proof electrodes W.

In some embodiments, as shown in FIG. 26, the temperature sensor F is a wire wound resistor. The winding resistor is a wire extending in a broken line pattern, and two ends of the wire are respectively electrically connected with the two fool-proof electrodes W (with reference to FIG. 25). Through a winding design, a temperature sensing area of the temperature sensor F may be enlarged, which improves a temperature sensing precision thereof.

In some embodiments, as shown in FIG. 27, one fool-proof electrode of the two fool-proof electrodes W is grounded. That is, the grounding electrode 10 is multiplexed as a fool-proof electrode W, which saves a design space for specially forming a dedicated fool-proof electrode W.

In some embodiments, as shown in FIG. 28, the second substrate 2 is provided with a second through hole Ho2. An orthographic projection of the second through hole Ho2 on the first substrate 1 at least partially overlaps with a grounding fool-proof electrode W.

In the case where the first substrate 1 is assembled with the second substrate 2, the second substrate 2 is prone to blocking a connection path between the grounding fool-proof electrode W and the circuit board 202. By providing the second through hole Ho2, and setting the orthographic projection of the second through hole Ho2 on the first substrate 1 to partially overlap with the grounded fool-proof electrode W, an end of the grounding probe J5 is enabled to be in contact with the grounding pad T3 of the circuit board 202, and another end of the grounding probe J5 may sequentially pass through the first through hole Ho1 in the bottom U2 and the second through hole Ho2 in the second substrate 2, and then contacts the grounded fool-proof electrode W.

It will be noted that, FIGS. 24, 25 and 27 provided in the embodiments of the present disclosure only exemplarily show schematic diagrams of one or two circuits L′. In an actual product, the microfluidic chip 100 includes a plurality of circuits L′; and in each circuit L′, a driving electrode Q is electrically connected with a bonding electrode P.

In some embodiments, as shown in FIG. 28, the second substrate 2 is provided with at least one second window C2. The second window C2 is configured to expose the plurality of bonding electrodes P and/or the plurality of detection electrodes Z of the first substrate 1.

The foregoing descriptions are merely specific implementations of the present disclosure. However, the protection scope of the present disclosure is not limited thereto. Changes or replacements that any person skilled in the art could conceive of within the technical scope of the present disclosure shall be included in the protection scope of the present disclosure. Therefore, the protection scope of the present disclosure shall be subject to the protection scope of the claims.

1. A crimping device for a microfluidic chip, comprises:
a cover plate;
a bottom plate, assembled with the cover plate, wherein the bottom plate is provided with a carrying recess, a mouth of the carrying recess faces the cover plate, and a bottom of the carrying recess is provided with an opening; and
at least one probe assembly, including a plurality of probes, wherein the probe assembly is fixedly connected with the bottom of the carrying recess; ends, proximate to the cover plate, of the plurality of probes are configured to be in contact with the microfluidic chip; and ends, away from the cover plate, of the plurality of probes pass through the opening.

2. The crimping device according to claim 1, wherein the probe assembly further includes:
a needle module, fixedly connected with the bottom of the carrying recess, wherein the needle module is provided therein with a plurality of mounting holes; and a probe gf the plurality of probes passes through a respective mounting hole, and is fixed in the mounting hole.

3. The crimping device according to claim 1, wherein the bottom of the carrying recess includes a plurality of carrying portions, the plurality of carrying portions are arranged at intervals around a wall of the carrying recess, and at least one of the plurality of carrying portions is raised with respect to the wall; and
the probe assembly is disposed between two adjacent carrying portions; and two ends of the probe assembly are fixedly connected with the two adjacent carrying portions, respectively.

4. The crimping device according to claim 1, wherein the at least one probe assembly includes a plurality of probe assemblies; and
at least one of the plurality of probe assemblies is a bonding probe assembly; the bonding probe assembly is disposed proximate to a wall of the carrying recess; and ends, proximate to the cover plate, of a plurality of probes of the bonding probe assembly are configured to be in contact with bonding electrodes of the microfluidic chip; or
the at least one of the plurality of probe assemblies is the bonding probe assembly; the bonding probe assembly is disposed proximate to the wall of the carrying recess; and the ends, proximate to the cover plate, of the plurality of probes of the bonding probe assembly are configured to be in contact with the bonding electrodes of the microfluidic chip; another at least one of the plurality of probe assemblies is a driving probe assembly; with respect to the bonding probe assembly, the driving probe assembly is disposed away from the wall of the carrying recess; and ends, proximate to the cover plate, of a plurality of probes of the driving probe assembly are configured to be in contact with driving electrodes of the microfluidic chip.

5. (canceled)

6. The crimping device according to claim 1, wherein the bottom of the carrying recess is further provided therein with a first through hole; and
the crimping device further comprises
a grounding probe, wherein the grounding probe is fixedly connected with the bottom of the carrying recess; an end, proximate to the cover plate, of the grounding probe is configured to be in contact with the microfluidic chip; and an end, away from the cover plate, of the grounding probe passes through the first through hole; and/or
the crimping device further comprises a press-fit structure, disclosed on a surface of the cover plate facing the bottom plate, wherein the press-fit structure has elasticity; in a case where the cover plate is buckled with the bottom plate, an orthographic projection of the press-fit structure on a reference surface at least partially overlaps with an orthographic projection of the probe assembly on the reference surface, wherein the reference surface is a plane where a surface, proximate to the cover plate, of the bottom of the carrying recess is located; and/or
the crimping device further comprises hinge structures, each including a first hinge and a second hinge movably connected with each other, wherein the first hinge is fixedly connected with the cover plate, and the second hinge is fixedly connected with the bottom plate.

7. The crimping device according to claim 6, wherein in a case where the crimping device comprises the grounding probe, the plurality of probes and the grounding probe have elasticity in respective length extension directions thereof; and
an elastic deformation range of the grounding probe is greater than an elastic deformation range of at least one of the plurality of probes.

8. (canceled)

9. The crimping device according to claim 6, wherein in a case where the crimping device comprises the Dress-fit structure, the press-fit structure includes a press-fit plate and an elastic member; the elastic member is located between the press-fit plate and the cover plate; an end of the elastic member is connected with the press-fit plate, and another end of the elastic member is connected with the cover plate; in the case where the cover plate is buckled with the bottom plate, an orthographic projection of the press-fit plate on the reference surface at least partially overlaps with the orthographic projection of the probe assembly on the reference surface.

10. (canceled)

11. The crimping device according to claim 6, wherein in a case where the crimping device comprises the hinge structures, the crimping device further comprising:
a snap-fit structure including a buckle and a latching slot, wherein the buckle is fixedly connected with one of the cover plate and the bottom plate, and the latching slot is provided in another one of the cover plate and the bottom plate; and the buckle is capable of being snap-fitted to the latching slot; and
the hinge structures and the snap-fit structure are disposed on two opposite sides of the cover plate and the bottom plate, respectively.

12. A detection apparatus for a microfluidic chip, comprising:
the crimping device according to claim 1;
a circuit board, disposed on a side of the bottom plate of the crimping device away from the cover plate, wherein the circuit board includes a substrate and a plurality of pads disposed on the substrate, and a pad of the plurality of pads is in contact with a respective probe of the crimping device; and
a processor, disposed on the circuit board and electrically connected with the plurality of pads, wherein the processor is configured to transmit detection signals to probes in contact with the pads through the pads, receive feedback signals from the probes, and process the feedback signals.

13. The detection apparatus according to claim 12, wherein the plurality of pads are disposed on a side of the substrate proximate to the bottom plate, and the processor is disposed on a side of the substrate away from the bottom plate; and/or
the bottom of the carrying recess is further provided therein with a first through hole; the crimping device includes a grounding probe; the grounding probe is fixedly connected with the bottom of the carrying recess; an end, proximate to the cover plate, of the grounding probe is configured to be in contact with the microfluidic chip; and an end, away from the cover plate, of the grounding probe passes through the first through hole; the circuit board further includes a grounding pad; and the grounding pad is in contact with the grounding probe; and/or
the circuit board further includes a resistor, and the resistor is connected in parallel with the processor.

14-16. (canceled)

17. A detection system for a microfluidic chip, comprising:
the detection apparatus according to claim 12; and
an industrial personal computer, electrically connected with the circuit board of the detection apparatus.

18. A method for detecting a microfluidic chip, in which a detection being performed by using the detection apparatus according to claim 12;
the method comprising:
placing the microfluidic chip in the carrying recess of the bottom plate of the detection apparatus, wherein the microfluidic chip includes a plurality of electrodes, and an electrode of the plurality of electrodes is in contact with a respective probe of the detection apparatus;
detecting whether two electrodes, insulated from each other, of the plurality of electrodes are shorted to each other; and/or
the microfluidic chip including a plurality of circuits each having at least two of the plurality of electrodes connected in series, detecting whether a circuit of the plurality of circuits is broken.

19. The method according to claim 18, wherein detecting whether the two electrodes, insulated from each other, of the plurality of electrodes are shorted to each other, includes:
combining the plurality of electrodes pairwise to obtain a plurality of electrode pairs, wherein two electrodes of each electrode pair are insulated from each other; and for any two electrode pairs, two electrodes of one electrode pair are not completely same as two electrodes of another electrode pair;
detecting whether the two electrodes of each electrode pair are shorted to each other;
recording positions of two electrodes of an electrode pair according to that the two electrodes of the electrode pair are shorted to each other; and
determining that the microfluidic chip has no short-circuit fault according to that the two electrodes of each of the plurality of electrode pairs are not shorted to each other.

20. The method according to claim 19, wherein detecting whether the two electrodes of each electrode pair are shorted to each other, includes:
detecting a voltage between the two electrodes of the electrode pair;
determining that two electrodes of an electrode pair are shorted to each other according to that a voltage is less than or equal to a threshold voltage; and
determining that two electrodes of an electrode pair are not shorted to each other according to a voltage is greater than the threshold voltage.

21. (canceled)

22. The method according to claim 18, wherein detecting whether the circuit of the plurality of circuits is broken, includes:
detecting a voltage between two electrodes, at two ends, of at least two electrodes connected in series with each other of the circuit;
determining that the circuit is broken according to that the voltage is greater than a threshold voltage, and recording positions of the at least two electrodes connected in series with each other of the circuit; and
determining that the circuit is non-broken according to the voltage is less than or equal to the threshold voltage.

23. The method according to claim 18, wherein the plurality of electrodes includes two fool-proof electrodes, the two fool-proof electrodes are electrically connected with each other, and the two fool-proof electrodes are arranged asymmetrically with respect to a setting center line of the microfluidic chip, wherein the microfluidic chip includes a first side edge and a second side edge that are opposite to each other, and the setting center line is a center line parallel to the first side edge and the second side edge of the microfluidic chip; and
the method further comprises:
detecting whether two electrodes at target positions are shorted to each other, wherein the target positions are positions where the two fool-proof electrodes are located in a case where the microfluidic chip is placed in the detection apparatus in a correct position;
determining that the two electrodes at the target positions are not the two fool-proof electrodes according to that the two electrodes at the target positions are not shorted to each other, and reversing positions of the first side edge and the second side edge of the microfluidic chip; and
determining that the two electrodes at the target positions are the two foolproof electrodes and the microfluidic chip is placed in the correct position, according to that the two electrodes at the target positions are shorted to each other.

24. A method for manufacturing a microfluidic chip, comprising:
forming a first substrate and a second substrate of the microfluidic chip, the first substrate including a plurality of electrodes;
placing the first substrate in a detection apparatus, and detecting the first substrate by the method according to claim 18;
forming a dielectric layer on the plurality of electrodes of the first substrate according to a detection result that no fault exists;
detecting the first substrate on which the dielectric layer has been formed;
forming a first hydrophobic layer on the dielectric layer according to a detection result that no fault exists;
detecting the first substrate on which the first hydrophobic layer has been formed; and
assembling the first substrate on which the first hydrophobic layer has been formed with the second substrate, according to a detection result that no fault exists.

25. A microfluidic chip, comprising:
a first substrate, including two fool-proof electrodes, wherein the two fool-proof electrodes are electrically connected with each other, and arranged asymmetrically with respect to a setting center line of the microfluidic chip, wherein the microfluidic chip comprises a first side edge and a second side edge opposite to each other; the setting center line passes through midpoints of lines connecting the first side edge with the second side edge at arbitrary positions, and is parallel to the first side edge and the second side edge; and
a second substrate, assembled with the array substrate.

26. The microfluidic chip according to claim 25, wherein a fool-proof electrode of the two fool-proof electrodes is grounded; or
the fool-proof electrode of the two fool-proof electrodes is grounded; the second substrate is provided with a second through hole, and an orthogonal projection of the second through hole on the first substrate at least partially overlaps with the grounded fool-proof electrode; or
the first substrate includes a plurality of driving electrodes and a plurality of bonding electrodes, and a bonding electrode is electrically connected with at least one of the plurality of driving electrodes, wherein
two bonding electrodes of the plurality of bonding electrodes serve as the two fool-proof electrodes, and the two fool-proof electrodes are both disposed on a side of the setting center line proximate to the first side edge; or
the first substrate further includes a temperature sensor, and the temperature sensor is disposed adjacent to and insulated from at least one driving electrode, and the temperature sensor is electrically connected with the two fool-proof electrodes; or
the first substrate further includes the temperature sensor, and the temperature sensor is disposed adjacent to and insulated from the at least one driving electrode, and the temperature sensor is electrically connected with the two fool-proof electrodes; the temperature sensor is a wire wound resistor, the wound wire resistor is a wire extending in a broken line pattern, and two ends of the wire are electrically connected with the two fool-proof electrodes, respectively.

27-30. (canceled)

31. The microfluidic chip according to claim 25, wherein the first substrate further includes a plurality of driving electrodes, a plurality of bonding electrodes and a plurality of detection electrodes, and at least one driving electrode is electrically connected with both a bonding electrode and a detection electrode, wherein
the bonding electrode and the detection electrode electrically connected with the at least one driving electrode are configured to be electrically connected with a detection apparatus to realize an open-circuit detection.

32. (canceled)
